SEQUENCE LISTING (18 sequences)

SEQ ID NO: 1 - DNA, 2561 bp, Homo sapiens
  CDS (1)..(1290)
atggccgccaacatgtacagggtcggagactacgtctactttgagaactcctccagcaacccatacctgatccggagaatcgaggagctcaacaagacggccaatgggaacgtggaggccaaagtggtgtgcttctaccggaggcgggacatctccagcaccctcatcgccctggccgacaagcacgcaaccctgtcagtctgctataaggccggaccgggggcggacaacggcgaggaaggggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggttggagacccaggtgtgggaggcgcacaacccactcacagacaagcagatcgaccagttcctggtggtggcccgctctgtgggcaccttcgcacgggccctggactgcagcagctccgtccgacagcccagcctgcacatgagcgccgcagctgcctcccgagacatcaccctgttccacgccatggatactctccacaagaacatctacgacatctccaaggccatctcggcgctggtgccgcagggcgggcccgtgctctgcagggacgagatggaggagtggtctgcatcagaggccaaccttttcgaggaagccctggaaaaatatgggaaggatttcacggacattcagcaagattttctcccgtggaagtcgctgaccagcatcattgagtactactacatgtggaagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacatgtcgtctctgcgcatcttgttggacatattggaagaaatatggtggcttgaaaatgccaacccggttagatggagagaggccaggaccaaaccgcagtaacatgagtccccacggcctcccagcccggagcagcgggagccccaagtttgccatgaagaccaggcaggctttctatctgcacacgacgaagctgacgcggatcgcccggcgcctgtgccgtgagatcctgcgcccgtggcacgctgcgcggaacccctacctgcccatcaacagcgcggccatcaaggccgagtgcacggcgcggctgcccgaagcctcccagagcccgctggtgctgaagcaggcggtacgcaagccgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggcaacatgaagaagcgcctcttgatgcccagtaggggtctggcaaaccacggacagaccaggcacatgggaccaagccggaacctcctgctcaacgggaagtcctaccccaccaaagtgcgcctgatccgggggggctccctgcccccagtcaagcggcggcggatgaactggatcgacgccccgggtgacgtgttctacatgcccaaagaggagaccaggaagatccgcaagctgctctcatcctcggaaaccaagcgtgctgcccgccggccctacaagcccatcgccctgcgccagagccaggccctgccgccgcggccaccgccacctgcgcccgtcaacgacgagcccatcgtcatcgaggactaggggccgcccccacctgcggccgccccccgcccctcgcccgcccacacggccccttcccagccagcccgccgcccgcccctcagtttggtagtgccccacctcccgccctcacctgaagagaaacgcgctccttggcggacactgggggaggagaggaagaagcgcggctaacttattccgagaatgccgaggagttgtcgtttttagctttgtgtttactttttggctggagcggagatgaggggccaccccgtgcccctgtgctgcggggccttttgcccggaggccgggccctaaggttttgttgtgttctgttgaaggtgccattttaaattttatttttattactttttttgtagatgaacttgagctctgtaacttacacctggaatgttaggatcgtgcggccgcggccggccgagctgcctggcggggttggcccttgtcttttcacccgccc

SEQ ID NO: 2 - protein, 430 aa, Homo sapiens
MAANMYRVGDYVYFENSSSNPYLIRRIEELNKTANGNVEAKVVCFYRRRDISSTLIALADKHATLSVCYKAGPGADNGEEGEIEEEMENPEMVDLPEKLKHQLRHRELFLSRQLESLPATHIRGKCSVTLLNETESLKSYLEREDFFFYSLVYDPQQKTLLADKGEIRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAEAESKLKQVYIPNYNKPNPNQISVNNVKAGVVNGTGAPGQSPGAGRACESCYMSSLRILLDILEEIWWLENANPVRWREARTKPQ

SEQ ID NO: 3 - protein, 33 aa, Homo sapiens
MSSLRILLDILEEIWWLENANPVRWREARTKPQ

SEQ ID NO: 4 - DNA, 21 bp, Artificial Sequence
  Description of Artificial Sequence  Synthetic Primer
agctacgaggaggacaacggg

SEQ ID NO: 5 - protein, 33 aa, Mouse
MSPLRILVDILEEIWWLENANPVRWREAGTKPQ

SEQ ID NO: 6 - DNA, 20 bp, Artificial Sequence
  Description of Artificial Sequence  Synthetic Primer
cacgcttggtttccgaggat

SEQ ID NO: 7 - DNA, 22 bp, Artificial Sequence
  Description of Artificial Sequence  Synthetic Primer
aagaccaccgacagatacgtgc

SEQ ID NO: 8 - DNA, 21 bp, Artificial Sequence
  Description of Artificial Sequence  Synthetic Primer
tggcctctctccatctaaccg

SEQ ID NO: 9 - DNA, 45 bp, Artificial Sequence
  Description of Artificial Sequence  Synthetic Primer
gagagctgttacatgtcgtctgacatattggaagaaatatggtgg

SEQ ID NO: 10 - DNA, 43 bp, Artificial Sequence
  Description of Artificial Sequence  Synthetic Primer
gggcctgcgagagctgttaatgtcgtctctgcgcatcttgttg

SEQ ID NO: 11 - DNA, 43 bp, Artificial Sequence
  Description of Artificial Sequence  Synthetic Primer
caacaagatgcggagagacgacattaacagctctcgcaggccc

SEQ ID NO: 12 - DNA, 333 bp, Artificial Sequence
  Description of Artificial Sequence  Synthetic Primer
aagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacaccacacagtcttaccagtggtattcttggggtccccctaacatgcagtgtcgtctctgcgcatcttgttggacatattggaagaaatatggtggcttgaaaatgccaacccggttagatggagagaggcca

SEQ ID NO: 13 - DNA, 286 bp, Artificial Sequence
  Description of Artificial Sequence  Synthetic Primer
aagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacatgtcgtctctgcgcatcttgttggacatattggaagaaatatggtggcttgaaaatgccaacccggttagatggagagaggcca

SEQ ID NO: 14 - DNA, 32 bp, Artificial Sequence
  Description of Artificial Sequence  Synthetic Primer
atgccgtcaacargtacagggtcggagactac

SEQ ID NO: 15 - DNA, 21 bp, Artificial Sequence
  Description of Artificial Sequence  Synthetic Primer
tgggctctctccatctaaccg

SEQ ID NO: 16 - DNA, 45 bp, Artificial Sequence
  Description of Artificial Sequence  Synthetic Primer
ccaccatatttcttccaatatgtcagacgacatgtaacagctctc

SEQ ID NO: 17 - DNA, 43 bp, Artificial Sequence; identical to SEQ ID NO: 10
  Description of Artificial Sequence  Synthetic Primer

SEQ ID NO: 18 - DNA, 43 bp, Artificial Sequence; identical to SEQ ID NO: 11
  Description of Artificial Sequence  Synthetic Primer

(19) United States
(12) Patent Application Publication
(10) Pub. No.: US 2006/0204957 A1
(43) Pub. Date: Sep. 14, 2006

(54) Mta1s, a steriod hormone receptor corepressor

(76) Inventors: Rakesh Kumar, Houston, TX (US); Rui-An Wang, Houston, TX (US); Abhijit Mazumdar, Pearland, TX (US); Ratna Vadlamudi, San Antonio, TX (US)

Correspondence Address: FULBRIGHT & JAWORSKI L.L.P., 600 CONGRESS AVE., SUITE 2400, AUSTIN, TX, 78701, US

(21) Appl. No.: 10/513,423

(22) PCT Filed: May 1, 2003
(86) PCT No.: PCT/US03/13549

§ 371 Date: Apr. 27, 2006

Related U.S. Application Data
(60) Provisional application No. 60/377,562, filed on May 3, 2002.

(51) Int. Cl. C12Q 1/68; C07H 21/04; C12P 21/06; C07K 14/72; C07K 16/28

(52) U.S. Cl. 435/6; 435/69.1; 435/320.1; 435/325; 530/350; 530/388.22; 536/23.5

(57) ABSTRACT

The present invention concerns the use of methods and compositions to diagnose, treat and identify therapeutic molecules for cancer, in particular hormone insensitive cancers. The invention includes polypeptides and nucleic acids encoding polypeptides of MTA1s, a novel protein derived from alternative splicing of the MTA1 gene. Other embodiments include antibody compositions for the diagnosis and prognosis related to the aberrant expression of the MTA1s polypeptide.

This application claims priority to U.S. Provisional Patent application Ser. No. 60/377,562, which is incorporated herein by reference.

The government may own rights in the present invention pursuant to grant number CA80066, CA65746, and CA84456 from the National Institutes of Health and BCTR 2000-830-1 from the Susan G. Komen Breast Cancer Foundation.

### BACKGROUND OF THE INVENTION

I. Field of the Invention

The present invention relates generally to the fields of molecular biology and cancer therapy. More particularly, it concerns compositions and methods comprising isolated nucleic acids encoding MTA1s polypeptides, antibodies immunoreactive with MTA1s polypeptides, and isolated MTA1s polypeptides, as well as diagnostic, prognostic and therapeutic compositions and methods.

II. Description of Related Art

The nuclear hormone receptor superfamily includes receptors for thyroid and steroid hormones, retinoids and vitamin D, as well as different “orphan” receptors for unknown ligands. Ligands for these receptors can regulate gene expression by binding to nuclear receptors. Nuclear receptors act as ligand-inducible transcription factors by directly interacting as monomers, homodimers, or heterodimers with DNA response elements of target genes, as well as by “cross-talking” to other signaling pathways. The effects of nuclear receptors on transcription are mediated through recruitment of coregulators. A subset of receptors binds corepressor factors and actively represses target gene expression in the absence of ligand. Corepressors are found within multicomponent complexes that contain histone deacetylase activity. Deacetylation leads to chromatin compactation and transcriptional repression. Upon ligand binding, the receptors undergo a conformational change that allows the recruitment of multiple coactivator complexes. Some of these proteins are chromatin remodeling factors or possess histone acetylase activity, whereas others may interact directly with the basic transcriptional machinery. Recruitment of coactivator complexes to the target promoter causes chromatin decompactation and transcriptional activation. The characterization of corepressor and coactivator complexes, in concert with the identification of the specific interaction motifs in the receptors, has demonstrated the existence of a general molecular mechanism by which different receptors elicit their transcriptional responses in target genes.

Steroid hormones (SHs) are lipophilic molecules derived from cholesterol and synthesized in the adrenal cortex (glucocorticoids, mineralocorticoids, and adrenal androgens), the testes (testicular androgens, estrogen), and the ovary and placenta (estrogens and progestagens or progestins). SHs reach their target cells via the blood, where they are bound to carrier proteins, and because of their lipophilic nature pass the cell membrane by simple diffusion. Within the target cells SHs bind to steroid hormone receptors (SHRs), the key mediators of SH action. SHRs typically exert positive or negative effects on the expression of target genes. Binding of agonistic or antagonistic ligands leads to different allosteric changes of SHRs making them competent to exert positive or negative effects on the expression of target genes by different mechanisms. These mechanisms include liganded SHR-complexes binding to chromatin organized DNA sequences in the vicinity of target genes, termed hormone response elements (HREs) that typically initiates chromatin remodeling and activating or repressing signals to the target genes transcription machinery. Other mechanisms act through protein-protein interactions with other sequence-specific transcription factors and other non-genomic pathways such as protein kinase cascades in the cytoplasm. Still, in other mechanisms of action the SH response may be integrated with the intracellular signaling network via cross-talk of SHRs with signal transduction pathways, such as protein kinase cascades. SHRs modulate numerous responses in a large variety of cells depending on the physiological, cellular and genetic context.

Proliferation of breast and endometrial cells is under the control of ovarian steroid hormones (SHs) such as estrogen and progesterone. They mediate diverse physiological functions via interaction with nuclear-localized steroid hormone receptors (HRs). The SH receptor complex modifies the expression of SH-regulated genes by binding to conserved binding sites in their promoter region or through cross-talk with other transcription factors. In non-malignant tissues, HRs are in balance with other factors regulating proliferation, differentiation and apoptosis. While dysfunction of the regulatory mechanisms is a part of malignant transformation, functional SH receptors can promote growth of SH-responsive tumors. Therefore, anti-hormones that block the interaction of steroid hormones with the SH receptor are useful tools for the treatment of SH-responsive carcinomas. However, a portion of ER-positive breast cancers and most endometrial cancers do not respond to anti-estrogens and continued treatment results in hormone resistance, mostly without loss of the ER. (Flototto, et al., 2001).

Toh et al. (1994) analyzed a candidate metastasis-associated gene, Mta1, which was isolated by differential cDNA library screening using the 13762NF rat mammary adenocarcinoma metastatic system. Northern blot analyses showed that the Mta1 mRNA expression level was 4-fold higher in the highly metastatic cell line MTLn3 than in the nonmetastatic cell line MTC.4. The Mta1 gene was expressed in various normal rat organs, especially in the testis. The mRNA expression levels of the human homolog of the Mta1 gene, MTA1 (accession No. NM_{—}004689), also correlated with the metastatic potential in 2 human breast cancer metastatic systems. MTA1 was expressed as an approximately 3-kb transcript. The full-length human MTA1 cDNA sequence contains an open reading frame encoding a 715 amino acid protein (accession No. AAA78935) with several possible phosphorylation sites and a proline-rich amino acid stretch at the carboxyl-terminal end, matching a consensus sequence for the Src homology 3 domain-binding motif. The molecular mass of the MTA1 protein is approximately 80 kD. MTA1 has been shown to be a corepressor of nuclear Estrogen Receptor α (ER), and represses ER-driven transactivation (Mazumdar, et al., 2001). The invasiveness and metastatic potential of several human cancer cells have been shown to correlate with MTA1 expression.

None of the methods for diagnosis, prognosis, or treatment of hormone insensitive diseases or metastatic cancer are entirely satisfactory, thus new and improved methodologies are needed.

### SUMMARY OF THE INVENTION

The present invention includes compositions comprising and methods for using isolated MTA1s polypeptides and/or nucleic acids encoding MTA1s polypeptides, (GenBank accession number AF508978) and antibodies immunoreactive with MTA1s polypeptides, as well as diagnostic, prognostic and therapeutic compositions and methods.

In certain embodiments an isolated polypeptide may comprise the amino acid sequence of MTA1s. The isolated polypeptide may comprise 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, or 33 consecutive carboxy amino acids of MTA1s (amino acids 398 to 430 of SEQ ID NO:2 and SEQ ID NO:3). Other variants of the peptides, proteins, or polypeptides of the invention are contemplated and may be derived by one of skill in the art in light of the description of the invention provided herein.

Other embodiments of the invention may include an antibody or antibodies that are immunoreactive to an MTA1s polypeptide (SEQ ID NO:2) and in particular amino acids 398 to 430 of SEQ ID NO:2. In certain embodiments the antibodies will selectively interact with MTA1s polypeptides and generally not interact with MTA1 polypeptides. Selective interaction does not exclude some reactivity to MTA1 polypeptides, but the interaction with MTA1s will be such that the affinity for it is distinguishable over any cross-reactivity with MTA1. The antibodies of the invention may selectively, preferentially, or specifically bind to MTA1s The antibodies may react with epitopes that include amino acids with the peptide segment from 398 to 430 of SEQ ID NO:2 and may include other amino acid sequences outside of the segment. The amino acids comprising an epitope may be in consecutive order or may be in non-consecutive order. Non-consecutive amino acid may form an epitope as a result of the conformation of the polypeptide or a portion thereof. The amino acid sequence differences between MTA1s and MTA1 are such that differentiation between the polypeptides, the characteristics of the polypeptides, and the antibodies used to detect them may be distinct. Antibodies may be immunoreactive to various epitopes of MTA1s polypeptide that include, but are not limited to 1, 2, 3,4, 5, 6, 7, 8, 9, 10, 11, 12, 13, 14, 15, 16, 17, 18, 19,20, 21, 22, 23, 24, 25, 26, 27, 28, 29, 30, 31, 32, or 33 amino acids of amino acids 398 to 430 of the full length MTA1s polypeptide (SEQ ID NO:2). The antibody or antibodies may be a monoclonal or polyclonal antibody preparation. An antibody composition of the present invention may be immunoreactive with MTA1s peptide, protein, or polypeptide. Antibody compositions may not be substantially immunoreactive to other human polypeptides or may preferentially recognize MTA1s, the antibody composition may bind other proteins, but binding of MTA1s is distinguishable from the immunoreactive binding of non-MTA1s molecules. In Addition, the antibody compositions are typically not immunoreactive to MTA1 polypeptides or in the least may be distinguish from MTA1 binding antibodies by intensity, localization, competition, affinity or the like. In certain embodiments the antibody compositions of the present invention may further comprises a detectable label. A detectable label may include, but is not limited to a fluorescent label, a chemiluminescent label, a radiolabel and an enzymatic label.

In yet other embodiments, hybridoma cells that produce a monoclonal antibody, which are immunoreactive to a MTA1s polypeptide are contemplated. The hybridoma cell may produce an antibody that is not immunoreactive to other human and/or MTA1 polypeptides. In still other embodiments an antisera composition comprising a polyclonal antisera, antibodies of which are preferentially or specifically immunoreactive to MTA1s are contemplated. An antisera may be derived from an animal other than a human. The antisera may be derived from animals including, but not limited to mouse, goat, horse, or rabbit. In particular embodiments an antisera is typically derived from a rabbit.

In still other embodiments of the invention include an isolated nucleic acid comprising a region, or the complement thereof, encoding an MTA1s polypeptide. In particular embodiments the nucleic acid is a human nucleic acid. A nucleic acid may be a complementary DNA or RNA. Nucleic acids of the present invention maybe a nucleic acid that is a complementary DNA. A nucleic acid of the present invention may comprise a promoter operably linked to the region, or the complement thereof, encoding the MTA1s polypeptide. The nucleic acid may also include a polyadenylation signal operably linked to the region encoding the MTA1s polypeptide as well as an origin of replication. In certain embodiments the nucleic acid may be a viral vector. Viral vectors may include, but are not limited to retrovirus, adenovirus, herpesvirus, vaccinia virus and adeno-associated virus. Any known viral expression construct may be used. The nucleic acid may be packaged in a virus particle, a liposome, or another DNA delivery vector.

In particular embodiments of the invention the present invention may include an isolated oligonucleotide comprising a sequence of nucleotides that include an alternative splice junction of the MTA1 gene that results in the MTA1s cDNA, or complement thereof. An oligonucleotide may also comprise the sequence of the novel segments of the MTA1s nucleic acid. The isolated oligonucleotide may comprise between about 5 to about 100, about 10 to about 75, about 15 to about 50, about 20 to about 25 consecutive bases that include or span the splice junction resulting in the MTA1s cDNA. The oligonucleotide may be complementary to the human or mouse MTA1s nucleic acid. An oligonucleotide may further comprise a detectable label, such as a fluorescent label, a chemiluminescent label, a radiolabel, an enzymatic label, or other known detectable labels used to identify, quantify, select, and/or amplify a nucleic acid.

Certain embodiments of the invention include oligonucleotides. Certain embodiments of the invention may be used in the context of an oligonucleotide pair that hybridize at positions along a nucleic acid that are approximately 10, 20, 30, 40, 50, 60, 70, 80, 90, 100, 200, 300, 400, 500, 1000, 2000 or more nucleotides, as well as nucleotide sequence with lengths there between, from the alternative splice junction resulting in MTA1s nucleotide sequence. Preferably the splice junction is flanked or spanned by the oligonucleotide pair. The oligonucleotide pair may include about 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more consecutive bases of MTA1s, as well as length there between. An oligonucleotide pair may be used in nucleic acid amplification reactions and may amplify a nucleic acid segment that includes the splice junction results in a MTA1s nucleic acid.

Other embodiments of the invention include expression vectors comprising a nucleic acid encoding a negative modulator of MTA1s. Negative modulators of MTA1s are molecules that inhibit or decrease the binding or activity of MTA1s in a cell, including MTA1s interaction with other proteins. Negative modulators include, but are not limited to anti-sense nucleic acids, ribozymes, intrabodies, and aptamers. Expression vectors may further comprising a promoter operatively positioned with respect to the nucleic acid encoding a negative modulator of MTA1. Promoters include, but are not limited to CMV IE, SV40 IE, RSV, β-actin, tetracycline regulatable promoter and ecdysone regulatable promoter. Expression vectors may also include a polyadenylation signal, such as polyadenylation signals from BGH, thymidine kinase or SV40.

Still other embodiments include host cells that express the MTA1s encoding nucleic acid or the MTA1s polypeptide or derivatives thereof. A host cell may be a stably or transiently transfected cell. MTA1s expression may be constitutive or inducible.

Certain embodiments include methods of detecting MTA1s expression. In particular, methods of the invention may include detecting MTA1s expression in a cell. Exemplary methods include contacting a cell with an antibody composition that is immunoreactive with MTA1s and detecting the antibody composition that is immunoreactive with the cell. An antibody composition may comprise a monoclonal or polyclonal antibody or antibodies. The cell may be a hormone independent cell. The hormone independent cell may be a tumor cell. The cell may be present in a tissue sample. The tissue sample may be a biopsy sample. In addition, an antibody may comprise a detectable label, such as a fluorescent, an isotopic, a radioactive, or an enzymatic label.

Methods of the invention may also include restoring hormone sensitivity to a cell. Restoring hormone sensitivity to a cell may comprise contacting a cell with a negative modulator of MTA1s. The negative modulator may be expressed or localized on the cell surface or within the cell. A negative modulator of MTA1s may be a peptide, an antisense molecule, intrabody, aptamer, or a small molecule.

Other embodiments include methods for inhibiting the growth of a cancer cell comprising contacting a cancer cell with a negative modulator of MTA1s. A negative modulator of MTA1s may be a peptide, an antibody, an intrabody, an aptamer, an anti-sense molecule, or a small molecule. A cancer cell may be within a subject and in particular a human subject.

In still other embodiments, methods of identifying modulators of MTA1s is contemplated. Exemplary methods of identifying a modulator of MTA1s typically comprise (i) providing a cell expressing a MTA1s polypeptide and a steroid hormone receptor responsive reporter construct; (ii) contacting the cell with a candidate substance; and (iii) comparing expression from the steroid hormone receptor responsive reporter construct when a candidate substance is added with the expression from a steroid hormone receptor responsive reporter construct observed when the candidate substance is not added, wherein an alteration in expression from a steroid hormone receptor responsive reporter construct indicates that the candidate substance is a modulator of MTA1s. A cell may be a tumor cell. In addition, the candidate substance may be a peptide, peptide mimetic or small molecule. In particular embodiments the candidate substance may be selected from a small molecule library. In alternative embodiments, a candidate substance may be a protein or peptide and may also be selected from a protein or peptide library. Alternatively, the candidate substance may be an MTA1s analogue.

Certain embodiments of the invention may include a modulator of MTA1s activity identified according to methods that typically comprise (i) providing a cell expressing a MTA1s protein and a steroid hormone receptor responsive reporter construct; (ii) contacting the cell with a candidate substance; and (iii) comparing activity of MTA1s observed when the candidate substance is added to the cell with the activity of MTA1s observed when the candidate substance is not added, wherein an alteration in activity of MTA1s indicates that the candidate substance is a modulator of the activity of MTA1s. The MTA1s activity to be modulated may be the repression of steroid receptor mediated transcription. In particular, the steroid receptor mediated transcription may be Estrogen receptor mediated transcription.

In yet other embodiments diagnostic and prognostic methods are contemplated. Exemplary methods may comprise i) obtaining a tissue sample from the subject; ii) determining the level of MTA1s in the tissue sample; iii) comparing the level of MTA1s in the tissue sample to a normal standard control, wherein an increased level of MTA1s is diagnostic for hormone insensitivity. The level of MTA1s may be determined by RNA analysis such as RNAse protection assay, real time polymnerase chain reaction, RNA blotting and the like. Alternatively, the level of MTA1s may be determined by protein analysis, such as immunohistochemistry, western blot analysis, immunoprecipitation, and the like. Prognostic methods may include i) providing a sample of tissue isolated from a cancerous tissue from a subject; ii) quantifying expression of MTA1s in the sample; and iii) correlating the quantity of expression of the MTA1s in the sample with a prognosis of the cancer in the subject, wherein higher expression of the MTA1s protein in the sample correlates with increased likelihood of a poor prognosis. The method of quantifying expression of MTA1s in the sample may be by RNA analysis, protein analysis as described herein or is well known.

In a particular embodiment, a method for detecting an MTA1s mRNA is contemplated. Exemplary methods may include the detection of the alternative splice junction of MTA1s that results in the production of MTA1s mRNA. Detection of the MTA1s mRNA may be by RNA analysis, such as RNase protection assay, DNA amplification, and RNA blotting. The mRNA may be isolated from a cell, a cancer cell, or a tissue comprising at least one normal or cancer cell.

Other embodiments of the invention contemplate methods for modulating non-genomic effects of hormone receptor sequestration in the cytoplasm by MTA1s. Non-genomic effects may include the modulation of protein kinase pathways. Exemplary methods of modulating non-genomic effects of steroid hormone receptor sequestration in the cytoplasm comprising contacting a cell with a modulator of MTA1s, such as a negative modulator of MTA1s and/or a peptide, polypeptide, protein, or small molecule. In certain embodiments, a negative modulator may be an antisense molecule, an intrabody, an aptamer, or a small molecule.

In still other embodiments methods of detecting MTA1s encoding mRNA is contemplated. Exemplary methods for detecting mRNA encoding a MTA1s protein include i) obtaining a tissue sample from a patient; ii) contacting nucleic acids isolated from a tissue sample with at least one nucleic acid sequence probe that is complimentary to and specifically hybridizes to the nucleic acid sequence encoding the MTA1s protein to form a hybridization product; and iii) detecting the increased presence of a hybridization product. A tissue sample may be isolated from breast, skin, prostate, ovary, uterus, cancer, or other tissues of a subject. Cancer tissue may be breast cancer, skin cancer, prostate cancer, ovarian cancer, uterine cancer, or other cancers that may or may not be hormone insensitive. A nucleic acid probe for use in methods described herein may include at least 5, 6, 7, 8, 9, 10, 11, 12, 13, 14, 15, 16, 17, 18, 19, 20 or more contiguous nucleotides that span the alternative splice junction of a nucleic acid encoding MTA1s. The patient or subject may be a human. The cancer may be breast cancer. The contacting of a tissue sample may be in situ or in vivo.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1J. MTA1s is a novel naturally occurring variant of MTA1. FIG. 1A is a schematic representation of the MTA1 clones. FIG. 1B illustrates an example of in vitro transcription and translation of T7-tagged MTA1 clones. FIG. 1C illustrates an example of expression of T7-MTA1 and T7-MTA1s cDNA as detected by an anti-T7 mAb. FIG. 1D illustrates an exemplary RT-PCR amplification of MTA1s open reading frame from SKBR-3 cells. FIG. 1E illustrates an example of the repression of E₂-induced ERE-luciferase activity by MTA1 clones in MCF-7 cells. FIG. 1F illustrates an example of the generation of the human MTA1s transcript by alternative splicing. FIG. 1G is an example of the MTA1s sequence showing the unique 33 amino acids in bold. FIG. 1H illustrates an example of the genomic organization of the C-terminal region of mouse MTA1. Black box, exons. Hatched box, exon deletion by cryptic splicing. FIG. 1I illustrates an exemplary RT-PCR (upper) and Southern blot (lower) analysis of RNA from MDA-MB435 cells. FIG. 1J shows the sequence of the mouse MTA1s C-terminal amino acids. The bold amino acid are amino acids that differ from human MTA1s C-terminus.

FIGS. 2A-2E. Expression of MTA1s protein. FIG. 2A illustrates the characterization of an exemplary MTA1s antibody. FIG. 2B illustrates an example of the expression of MTA1s and MTA1 in breast cancer cell lines. FIG. 2C illustrates exemplary levels of HER2, MTA1, and MTA1s proteins in MCF-7/HER2, MCF-7 and SKBR3 cells. FIG. 2D illustrates an example of the expression of MTA1 and MTA1s by RT-PCR. FIG. 2E illustrates an example of the expression of MTA1s during different developmental stages (shown in days) of the mammary gland. Virgin, 12 weeks. Lane 7, positive control lysate from T7-MTA1s transfected cells. Vinculin, loading control.

FIGS. 3A-3H. MTA1s typically localizes in the cytoplasm and interacts with ER. FIG. 3A illustrates an example of confocal microscopy of MCF-7 cells transiently transfected with T7-tagged MTA1 or MTA1s constructs. FIG. 3B illustrates exemplary expression of T7-MTA1s in ZR75 clones. FIG. 3C illustrates exemplary nuclear and cytoplasmic fractions that were assessed by immunoblotting with the indicated antibodies. FIG. 3D shows an example of the analysis of ZR75 clones that were treated with E₂ (10⁻⁹ M) for 30 min and the expression of T7-MTA1s or ERα analyzed by confocal microscopy. FIG. 3E shows an example of lysates from ZR75 clones were IP using T7-mAb and immunoblotted with the indicated antibodies. FIG. 3F illustrates exemplary GST pull-down assays. FIG. 3G shows an example of MTA1s interactions with GST-fusion proteins of the ER domains. FIG. 3H illustrates an example of expression of MTA1s and ERα in indicated cells by confocal microscopy.

FIGS. 4A-4D. MTA1s motif typically required for interaction and sequestration of ER. FIG. 4A illustrates an example of the effect of MTA1s, MTA1sₛₜₒₚ or MTA1s_{del} on ERE-dependent transcription in MCF-7 cells. FIG. 4B illustrates exemplary two-hybrid interactions. FIG. 4C illustrates an exemplary GST pull-down assays. Lane 1, 1% input. FIG. 4D shows an example of lysates from MCF-7 cells expressing either the vector or T7-MTA1s, T7-MTA1sₛₜₒₚ or T7-MTA1s_{del} were immunoprecipitated (IP) using T7-mAb and immunoblotted with ERα or T7 mAbs.

FIGS. 5A-5F. MTA1s may impair genomic responses and upregulates the MAP kinase pathway in ZR75R breast cancer cells. FIG. 5A illustrates the expression of estradiol target genes, pS2, c-Myc and Cathepsin D mRNAs. FIG. 5B illustrates an example of the status of E₂ (10⁻⁹ M for 16 h)-induced stimulation of pS2 mRNA. FIG. SC shows exemplary cell lysates that were EP with GRB2 antibody and immunoblotted as shown. Treatment, 5 min. with E₂ (10⁻⁹ M) or ICI 182780 (10⁻⁸ M) or both. FIG. 5D shows exemplary cell lysates from exponentially growing cells that were IP with GRB2 antibody and immunoblotted as shown. FIG. 5E shows exemplary cell lysates from exponentially growing cells were immunoblotted as shown. FIG. 5F shows exemplary cell lysates that were immunoblotted as shown. Treatment, 5 min with E₂ (10⁻⁹ M) or ICI 182780 (10⁻⁸ M) or both. Relative levels of p42/44Phos are shown underneath.

FIGS. 6A-6B. Typical transcriptional repression by MTA1s. FIG. 6A illustrates an example of repression of dexamethasone (10⁻⁹ M)-mediated stimulation of GRE-CAT activity in MCF-7 cells by MTA1s. FIG. 6B illustrates the exemplary repression of progesterone (10⁻⁹ M)-mediated stimulation of PRE-luciferase activity in MCF-7 cells by MTA1s. Cells were cotransfected with β-galactosidase to assess transfection efficiency, and normalized results are presented here.

FIGS. 7A-7B. Exemplary RNase protection analysis of MTA1s. FIG. 7A shows the sequence of the MTA1 and MTA1s RNase protection probes. FIG. 7B illustrates an exemplary RNase protection assay using the probes cloned from PCR reactions. Since MTA1s has a deletion of 47 bp, binding of the MTA1 probe to MTA1 transcript is expected to produce a 332 bp protected band while binding to MTA1s will generate two fragments of 201 and 84 bp sizes due to cleavage by RNAse. As expected, the MTA1 specific probe protected a band of 332 bp and also 201 and 84 bp bands (lanes 3 and 4). While in a reverse experiment, MTA1s probe protected a 285-bp MTA1s specific fragment and MTA1 was cleaved into 201 and 84 bp bands (lane 6). The MTA1s plasmid protected bands were used as a positive control for MTA1s bands (lane 5). Lane 1 and 2 shows the original size of the MTA1 and MTA1s probes.

FIG. 8. An example of the expression of MTA1 and MTA1s in various murine organs. Actin was used as a loading control. Lower Panel, quantitation of the levels of MTA1 and MTA1s relative to actin expression. Elevated levels of MTA1s protein were expressed in the brain, ovaries, adrenal glands, and virgin mammary glands. In contrast, MTA1s protein levels were extremely low in the testes, salivary and pituitary glands. The expression profile of MTA1s was quantitatively distinct from that of MTA1. For example, MTA1 was expressed in the pituitary gland whereas MTA1s was barely detectable. In contrast, MTA1s expression in the adrenal glands was elevated whereas the expression level of MTA1 was low in testes, and salivary and pituitary glands.

FIGS. 9A-9B. An example of MTA1s interaction with the ER in a yeast-based two-hybrid assay. FIG. 9A shows a schematic representation of ERα and MTA1s constructs used in a yeast-based two-hybrid system. FIG. 9B illustrates exemplary two-hybrid interactions. Yeast strain AH109 was transformed with plasmids carrying GAD-ERα, GBD-MTA1s full, GBD-MTA1s-Nter, and GBD-MTA1s-C-ter either alone or in combination. Colonies were initially selected for Leu Trp plates and streaked in quadruple minus plates Ade His Leu Trp to select for interaction. βGal assay was performed using filter-lift assay.

FIGS. 10A-10B. Exemplary illustration that MTA1s localizes in the cytoplasm and interacts with GR. FIG. 10A illustrates that MTA1s will typically sequester GR in the cytoplasm. ZR/pcDNA and ZR/MTA1s cells were treated with dexamethasome (10⁻⁷ M) for 30 min and expression of T7-MTA1s and GR was analyzed by confocal microscopy using antibodies against MTA1s or GR. FIG. 10B illustrate an example of MTA1s interaction with the GR in vivo. Lysates from ZR/pcDNA and ZR/MTA1s cells were immunoprecipitated using T7-mAb and immunoblotting with the indicated antibodies.

FIGS. 11A-11E. MTA1s may stimulate aggressive phenotypes in breast cancer cells. FIG. 11A illustrates an example of MTA1s overexpression inhibiting PRE-driven but not RARE-driven transcription. FIG. 10B illustrates the Growth-rate of ZR/pcDNA or ZR/MTA1s cells in monolayer culture in the presence of E₂ alone or with ICI 182780 (10⁻⁹ M for 4 days). FIG. 11C show an exemplary effect of MTA1s expression on anchorage independent growth of ZR/pcDNA and ZR/MTA1s cells grown in 10% serum in the presence or absence of ICI 182780 (10⁻⁹ M) or 4HT (10⁻⁸ M) for 14 days. FIG. 11D shows an exemplary effect of MTA1s expression on anchorage independent growth of MCF7/pcDNA and MCF7/MTA1s cells grown in 10% serum for 14 days. Insert shows the expression of T7-MTA1s. FIG. 11E illustrates that MTA1s may induce tumorigenesis of breast cancer cells. 5×10⁶ cells were subcutaneously inoculated in the athymic nude mice (n=10), and tumor volumes at day 21 were recorded.

FIGS. 12A-12B. MTA1s may upregulate the MAP kinase pathway in ZR75 breast cancer cells. FIG. 12A shows an example of ZR/pcDNA and ZR/MTA1s cells grown on Matrigel-coated coverslips were treated with E₂ (10⁻⁹ M) alone or with ICI 182780 (10⁻⁸ M) for 10 min. Cells were fixed, immunostained with phosphorylated-42/44 MAPK antibody or the actin binding protein phalloidin, and analyzed by confocal scanning microscopy. FIG. 12B illustrates an example of the quantitation of cells containing an increased cytoplasmic pool of activated MAPK.

FIGS. 13A-13B. An example of the expression of MTA1s in breast tumors. FIG. 13A shows exemplary levels of MTA1s and MTA1 proteins in paired tumor and adjacent normal tissues from the same patient. Ponceau staining was used for loading controls. N, Normal; and T, Tumor. Con (lane 9), positive control T-7 MTA1s transfected lysate. FIG. 13B shows an example of the expression of MTA1s, MTA1 and vinculin proteins in ER-positive and ER-negative breast tumors. Right panel, Quantitation of MTA1s expression after normalization to vinculin levels. The quantitation and normalization of the MTA1s signal to the control vinculin indicated a fourfold increase in MTA1s expression in the ER-negative tumors compared to the MTA1s level in ER-positive tumors. As with several anti-peptide antibodies against the ER coactivator, anti-MTA1s used here did not work well in assays of paraffin-embedded tumor specimens.

FIG. 14 shows exemplary proteins that have been identified as MTA1s binding proteins. The proteins include LIM 04, Casein Kinase 1-γ2, c-Myc, and an eukaryotic translation initiation factor 3.

FIG. 15 is a schematic of an exemplary assay for nuclear receptor function.

FIGS. 16A-16E FIG. 16A shows an exemplary yeast 2 hybrid analysis of the interaction between MTA1s and LMO4. FIG. 16B shows an exemplary interaction between MTA1s and synthesized LMO4. FIG. 16C and 16D shows an exemplary interaction between LMO4 and synthesized MTA1s. FIG. 16E shows an exemplary in vivo interaction between MTA1s and LMO4 by inmmunoprecipitation followed by western blotting.

FIG. 17 shows an example of recombinant MTA1 s interacting with APS.

FIGS. 18A-18D shows expression of MTA1s in endometrial tumors by immunohistochemistry (FIGS. 18A-18C) and expression of MTA1s as analyzed by western blotting (FIG. 18D)

FIG. 19 shows expression of MTA1s in endometrial tumorsform different grades by western blotting.

### DESCRIPTION OF ILLUSTRATIVE EMBODIMENTS

The present invention describes an isolated MTA1s, a novel protein derived form an alternatively spliced RNA transcript of the MTA1 gene, as well as providing for compositions comprising and methods of use for novel MTA1s nucleic acids, oligonucleotides, proteins, polypeptides, and peptides. In certain embodiments, compositions and methods are used in the diagnosis, prognosis and treatment of a variety of hormone independent diseases and metastatic cancers.

MTA1s was first cloned by analyzing numerous complementary DNAs (cDNA) of varying lengths from a human mammary gland cDNA library, as described in detail below. All of the MTA1 clones except the MTA1s clone were translated into proteins of expected sizes. The MTA1s clone was translated into a protein, which was smaller in size, 44 kDa rather than the approximately 80-kDa expected size, based on the MTA1s cDNA length.

The ability of MTA1s to repress the transactivating function of ER was investigated. The transient expression of MTA1s or full-length MTA1 but not other MTA1 cDNAs effectively blocked estradiol's ability to stimulate ERE transcription (see below). In addition, MTA1s clone also blocked the stimulation of the glucocortocoid responsive element (GRE) transcription by dexamethasone and progesterone responsive element (PRE) transcription by progesterone but not retinoic-acid responsive element (RARE) transcription.

A thorough analysis of the MTA1s sequence revealed a deletion of 47 bases between nucleotides ′1224 and +1272 of MTA1 (Genebank accession U35113), which probably is the result of an alternative splice producing a splice junction in the MTA1s cDNA at nucleotides 1192 and 1193 of SEQ ID NO:1 and the deletion of 47 nucleotides from the MTA1 mRNA. The MTA1s mRNA has been shown by the inventors to be expressed in normal brain, ovary, uterus, adrenal gland, lung, liver, salivary gland, testis and kidney. The alternative splicing of the MTA1 transcript causes a reading frame-shift, leading to a unique 33 amino acid carboxy terminal sequence that shows no significant homology with the sequences in the protein database, except for the presence of a potential steroid receptor binding motif LRILL. The novel 33 amino acid sequence of MTA1s is in the place of the carboxy terminal 318 amino acids of MTA1 resulting in a novel 430 amino acid protein, which lacks the carboxy terminal SH2 domain binding motif of the MTA1 protein. Comparison of the genomic sequences at the deletion site revealed that the 3′ donor site has a consensus site for splicing while 5′ has a non-consensus splicing acceptor site. Similar functional non-consensus splicing has been reported in other genes, such as various cell cycle genes.

MTA1s protein differs from MTA1 protein in a variety of characteristics that include mRNA sequence, amino acid sequence, cellular localization, and mechanisms of action. The MTA1s protein is a cytoplasmically localized protein that interacts with and sequesters SHRs in the cytoplasm. In contrast to the MTA1 protein, MTA1s localizes in the cytoplasm, directly interacts with various transcriptional activation domains of steroid receptors (e.g., the transcriptional activation function-2 domain of the ER), sequesters steroid receptors in the cytoplasm, and blocks expression of various steroid receptor target genes, as well as contributing to non-genomic effects, such as modulation of protein kinase cascades.

Several proteins have been identified that bind the carboxy terminal 33 amino acids of MTA1s by using yeast two hybrid screens using the carboxy terminus of MTA1s as bait. These protein interactions may provide some evidence of the role of MTA1s in hormonal-insensitivity, impairment of nuclear hormonal responses, participation in non-genomic functions of nuclear receptors, and modulation of signaling pathways in the cytoplasm. Several proteins have been identified that demonstrate binding with MTA1s. The proteins identified by the inventors as interacting with MTA1s include LIM 04 (clone S 99-2, Accession number NP_{—}00660), Casein Kinase 1, γ2 (clone S55-1, Accession number AAH20972), c-Myc Oncogene (clone S 36-1, Accession number GI:223833), Homologs to eukaryotic translation initiation factor 3, subunit 6 (48 kD) (Cone S 64-1, Accession number AAH00734) and ATP sulfurylase/adenosine 5′-phosphosulfate (APS) kinase (Genebank accession number NM_{—}005443).

LIM04 Domain—The LIM domain, an approximately 55-residue, cysteine-rich zinc-binding motif, is present in a variety of proteins including LIM homeobox (LHX) proteins that contain two LIM domains and one homeodomain. LHX genes are expressed in many types of neurons and other cell types, and deletion of LHX genes results in the loss of cell fate. Mice mutant for LHX1 have diminished organizer activity that results in lack of head structures anterior to rhombomere 3. In the central nervous system, development of forebrain and pituitary derivatives are defective in mice mutant for LHX2, LHX3, or LHX4, while activation of the LHX gene Isl1 is essential for the survival of motor neurons and neighboring interneurons. LMO2 represents a family of nuclear LIM-only (LMO) proteins that lack a DNA-binding homeodomain. Unregulated LMO2 expression induces T cell tumors, while deletion blocks hematopoietic development. The mechanism of LMO2 activity is thought to be the LIM domain-dependent assembly of transcription complexes and transcription regulation. LIM domains of nuclear proteins bind with high affinity to the widely expressed nuclear LIM interactor (NLI) and with lesser affinity to other transcription factors. Dimeric NLI supports assembly of heteromeric complexes of LIM proteins, and CHIP, the Drosophila ortholog of NLI, mediates enhancer-promoter interactions of the cut and ultrabithorax genes, presumably by complex formation with transcription factors.

Casein Kinase-1 γ2—Protein kinases CK1 (also known as casein kinase-1) are a family of monomeric enzymes ranging in size from 35 to 55 kDa. These enzymes have been found in all eukaryotes, from yeast to man, and are known to be present in the nucleus, cytoplasm and membrane fractions of cells Although CK1 is known as a Ser/Thr protein kinase, some species of yeast and Xenopus laevis have been shown to phosphorylate Tyr residues in synthetic peptides albeit with lower efficiency. A number of proteins have been reported to be phosphorylated by CK1 including: simian virus 40 large T antigen, the insulin receptor, a tumour necrosis factor receptor, the m-3 muscarinic acid receptor, inhibitor-2 of protein phosphatase-1 glycogen synthase, p53 and DARPP-32 . According to the structure of sites affected in casein fractions and consistent with subsequent studies with peptide substrates, CK1 was first classified as a phosphate-directed protein kinase, the consensus sequence of which is specified by a phosphorylated Ser or Thr residue at position n-3 upstream from the target amino acid (Sp/Tp-X—X—S). An individual Asp or Glu definitely proved unable to effectively replace the crucial phosphoserine acting as specificity determinant. Such a stringent requirement for a phosphorylated residue, however, was hardly consistent with a number of phosphoacceptor sites lacking this feature and nevertheless affected by CK1.

c-Myc—Mutations affecting the c-myc proto-oncogene are among the most common genetic lesions found in a variety of human and animal cancers. The c-Myc protein is a transcription factor that functions as an obligate heterodimer with its partner Max and binds the core recognition sequence CACGTG (E-box) and a number of related sequences. c-Myc can repress, as well as activate transcription. Under appropriate circumstances, both repression and overexpression of c-Myc can lead to apoptosis. Overexpression of c-Myc augments the apoptotic program and rapidly induces cell death when cells are deprived of survival factors. The tumor suppressor gene p53 has been implicated as a target of c-Myc regulation. c-Myc-induced apoptosis requires p53 in some but not all cases.

Eukaryotic Translation Initiation Factor 3. subunit 6—Eukaryotic translation initiation factor 3, subunit 6 (48 kD), is a fission Yeast Homolog of Murine Int-6 Protein, encoded by Mouse Mammary Tumor Virus Integration Site. It is associated with the conserved core subunits of Eukaryotic Translation Initiation Factor 3.

ATP sulfurylase/adenosine 5′-phosphosulfate (APS) kinase—

Sulfation reactions that are facilitated by a class of enzymes known as sulfotransferases. Sulfotransferases catalyze the transfer of a sulfate group from an activated donor onto a hydroxyl or an amino group of the acceptor molecule. The nucleotide-analogue 3′ phosphoadenosine 5′ phosphosulfate (PAPS) invariably serves as the activated sulfate donor (Klaassen and Boles, 1997). The sulfotransferases are functionally analogous to the kinases, which use adenosine 5′-triphosphate (ATP) as the activated phosphate donor. PAPS is generated from ATP and SO4⁻ by the sequential action of two enzymes: ATP sulfurylase, which synthesizes adenosine 5′ phosphosulfate (APS); and APS kinase, which adds an ATP-derived phosphate to the 3′ position of APS. Typically, both enzymatic activities are found within a single protein.

Two general classes of sulfotransferases exist: cytosolic sulfotransferases, which act on small molecule substrates, including steroids, neurotransmitters, and various metabolic end products (Falany, 1997); and the Golgi-localized, usually membrane-bound sulfotransferases which transfer sulfate onto protein tyrosine residues or carbohydrates on glycoproteins, proteoglycans, and glycolipids (Bowman and Bertozzi, 1999). Sulfation of metabolites by cytosolic sulfotransferases commonly leads to their inactivation or elimination by increasing water solubility and decreasing biological activity (Falany, 1997). By contrast, the spectrum of biological activities conferred onto their respective acceptors by the Golgi-resident sulfotransferases is much more diverse.

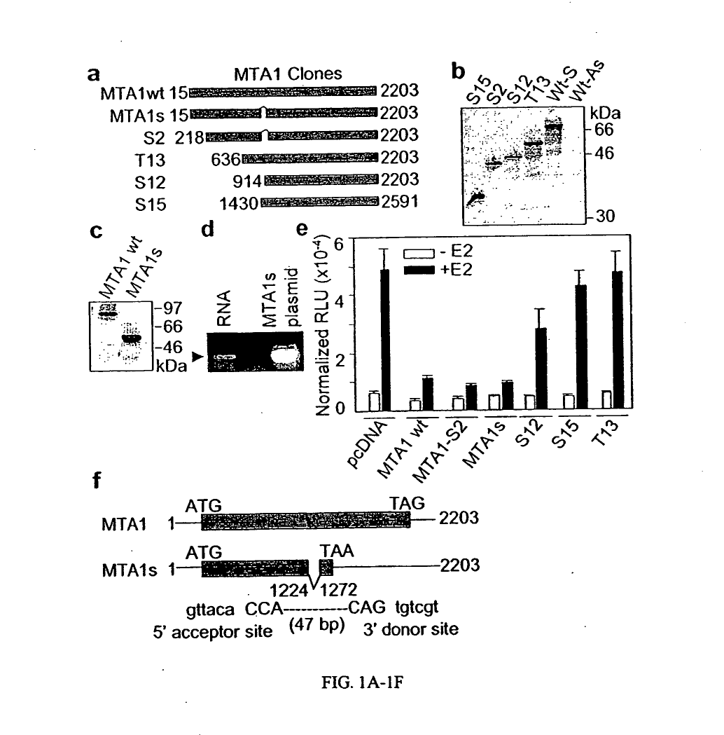

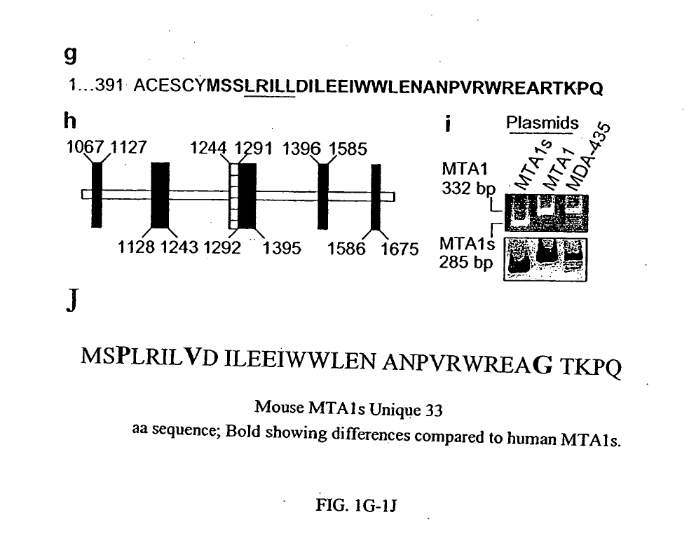

MTA1s compositions may participate in the modulation of these and other proteins in the cell. These findings indicate that MTA1s may be used as a diagnostic or prognostic marker in cancers and/or hormone insensitive cells. Other evidence in support of a regulatory role for MTA1s is that the 33 C-terminal amino acids show a high degree of conservation between mouse and human MTA1s proteins (FIG. 1J).

The inventors have discovered that HER2 overexpression lead to upregulation of MTA1s with concomitant sequestration of a portion of estrogen receptor in the cytoplasm. Clinically it is established that overexpression of HER2 oncogene in estrogen receptor-positive breast tumors counteract the effectiveness of anti-hormonal therapy. This indicates why anti-hormonal therapy fail to work in high HER2/ER+ cases. Therefore, targeting MTA1s may also potentiate the responsiveness of anti-hormonal and anti-HER2 therapies. The modulation of the activity or characteristics of MTA1s may be useful for the treatment of cancer, hornonally insensitive disease states or diseases resulting from a hormonal hypersensitivity.

Various embodiments of the invention include compositions comprising and methods of using antibodies immunoreactive with MTA1s. Antibodies of the present invention may be used as a diagnostic tool in the identification of cells, tissues, organs, or tissue masses that overexpress MTA1s. The methods may be diagnostic of metastatic potential, hormone insensitivity or other disorders. In certain embodiments, antibodies that specifically bind or are immunoreactive with MTA1s are produced. These antibodies may be polyclonal or monoclonal and may be used in a variety of immunologic based methods. Immunologic based assay include, but are not limited to immunohistochemistry, immunoprecipitation, ELISA, western blotting, in vivo imaging, and other methods and techniques utilizing immunologic compositions of the invention.

Other embodiments include compositions and methods utilizing isolated nucleic acids of MTA1s. Isolated nucleic acids of the invention may be used as diagnostic or therapeutic compositions. MTA1s specific nucleic acids may be used to identify the upregulation of MTA1s transcripts. Therapeutic application may entail the use of isolated nucleic acid of the invention as negative regulators or encode negative regualtors of MTA1s function or production.

Still other embodiments may utilize isolated MTA1s proteins, polypeptides, or peptides. In various embodiments, an isolated MTA1s protein, polypeptide, or peptide may be utilized. Compositions comprising an isolated MTA1s protein, polypeptide, or peptide include, but are not limited to antigenic, therapeutic, and diagnostic compositions and methods.

In various embodiments, the compositions and methods of the invention may be used to identify modulators of the MTA1s polypeptide or the transcription or modulation of the expression of the gene encoding MTA1s.

I. MTA1S Peptides and Polypeptides

MTA1s is a novel transcript and protein produced by the MTA1 gene. In addition to an entire MTA1s molecule, the present invention also relates to fragments of the polypeptides that may or may not retain various functions described below. Fragments, including the N-terminus of the molecule may be generated by genetic engineering of translation stop sites within the coding region (discussed below). Alternatively, treatment of the MTA1s with proteolytic enzymes, known as proteases, can produce a variety of N-terminal, C-terminal and internal fragments. Examples of fragments may include contiguous residues of SEQ ID NO:2 of 6, 7, 8, 9, 10, 11, 12, 13, 14, 15, 16, 17, 18, 19, 20, 21, 22, 23, 24, 25, 30, 35,40,45, 50, 55, 60, 65, 75, 80, 85, 90, 95, 100, 200, 300 or more amino acids in length, in so far as all or part of the novel 33 C-terminal amino acids of MTA1s (SEQ ID NO:3) are contained within the fragment. All or part of the C-terminal amino acids includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 amino acids of SEQ ID NO:3. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants of MTA1s

Amino acid sequence variants of the polypeptide can be substitutional, insertional or deletion variants. Deletion variants lack one or more residues of the native protein which are not essential for function or immunogenic activity. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+31.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule or in some cases inhibit or compete for interaction sites similar to the natural molecule. These principles may be used, in conjunction with the principles outline above, to engineer second generation molecules having many of the natural properties of MTA1s, but with altered and even improved characteristics. Furthermore these principles may be used to engineer inhibitory or competing peptides for the MTA1s protein.

B. Fusion Proteins

A specialized kind of insertional variant is the fusion protein. This molecule generally has all or a substantial portion of the native molecule, linked at the N— or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

C. Purification of Proteins

In certain embodiments, it may be desirable to purify MTA1s or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

D. Synthetic Peptides

The present invention also describes smaller MTA1s-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

The present invention also provides for the use of MTA1s proteins or peptides as antigens for the immunization of animals relating to the production of antibodies. It is envisioned that MTA1s, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

II. Antibodies Reactive with MTA1s.

Various embodiments of the invention (e.g., diagnostic and prognostic methods) will utilize compositions that preferentially or specifically identify MTA1s proteins or polypeptides. The present invention contemplates an antibody that is immunoreactive with a MTA1s molecule of the present invention, or any portion thereof containing the novel carboxy terminal 33 amino acids. An antibody can be a polyclonal or a monoclonal antibody. In a preferred embodiment, an antibody is a monoclonal antibody. Means for preparing and characterizing antibodies are well known in the art (see, e.g., Harlow and Lane, 1988, incorporated herein by reference).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of an antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the proteins and polypeptides of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to MTA1s-related antigen epitopes. Additionally, it is proposed that monoclonal antibodies specific to the particular MTA1s of different species may be utilized in other useful applications.

In general, both polyclonal and monoclonal antibodies against MTA1s may be used in a variety of embodiments. For example, they may be employed in antibody cloning protocols to obtain cDNAs or genes encoding other MTA1s. They may also be used in inhibition studies to analyze the effects of MTA1s related peptides in cells or animals. MTA1s antibodies will also be useful in immunolocalization studies to analyze the distribution of MTA1s during various cellular events, for example, to determine the cellular or tissue-specific distribution of MTA1s polypeptides under different points in the cell cycle. A particularly useful application of such antibodies is in purifying native or recombinant MTA1s, for example, using an antibody affinity column. The operation of all such immunological techniques will be known to those of skill in the art in light of the present disclosure.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, (e.g., a purified or partially purified MTA1s protein, polypeptide or peptide or cell expressing high levels of MTA1s). The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed. The spleen lymphocytes are obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmnunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

III. Nucleic Acids

The present invention also provides, in another embodiment, nucleic acids encoding MTA1s. The present invention is not limited in scope to these nucleic acids, however, as one of ordinary skill could, using these nucleic acids, readily identify related homologs in these and various other species (e.g., rat, rabbit, dog, monkey, gibbon, human, chimp, ape, baboon, cow, pig, horse, sheep, cat and other species).

In addition, it should be clear that the present invention is not limited to the specific nucleic acids disclosed herein. As discussed below, a “MTA1s encoding nucleic acid” may contain a variety of different bases and yet still produce a corresponding polypeptide that is functionally indistinguishable, and in some cases structurally, from MTA1s polypeptides disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the activity of MTA1s.

A. Nucleic Acids Encoding MTA1s

Nucleic acids according to the present invention may encode an entire MTA1s gene, a domain of MTA1s, or any other fragment of MTA1s as set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.”

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given MTA1s from a given species may be represented by natural variants that have slightly different nucleic acid sequences but, nonetheless, encode the same protein (see Table 1 below).

As used in this application, the term “a nucleic acid encoding a MTA1s” refers to a nucleic acid molecule that has been isolated free of total cellular nucleic acid. In preferred embodiments, the invention concerns a nucleic acid sequence as set forth in SEQ ID NO: 1 and comprises the alternative splice junction that produces a frameshift resulting in the MTA1s protein and a novel nucleic acid sequence. The term “as set forth in SEQ ID NO: 1” means that the nucleic acid sequence substantially corresponds to a portion of SEQ ID NO:1. The term “functionally equivalent codon” is used herein to refer to codons that encode the same amino acid, such as the six codons for arginine or serine (Table 1, below), and also refers to codons that encode biologically equivalent amino acids, as discussed in the following pages.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:1 and include the alternative splice junction are contemplated. Sequences that are essentially the same as those set forth in SEQ ID NO:1 and contain the alternative splice junction may also be functionally defined as sequences that are capable of hybridizing to a nucleic acid segment spanning the alternative splice junction containing the complement of SEQ ID NO:1 under standard conditions.

The DNA segments of the present invention include those encoding functionally equivalent MTA1s proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

TABLE 1

| | |
|---|---|
| Amino Acids | Codons |

| | | | |
|---|---|---|---|
| Alanine | Ala | A | GCA GCC GCG GCU |
| Cysteine | Cys | C | UGG UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGG GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAG AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UGA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

B. Oligonucleotide Probes and Primers

The present invention also encompasses DNA segments that are complementary, or essentially complementary, to the sequence set forth in SEQ ID NO:1, to sequences that span the alternative splice junction, or are within approximately 1000 nucleotides of the alternative splice junction. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein. Such sequences may encode entire MTA1s proteins or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used that are within 1000 nucleotides of the alternative splice junction, although others are contemplated. Longer polynucleotides encoding 250, 500, 1000, 1212, 1500, 2000, 2500, 3000 or 5000 bases and longer are contemplated as well. Such oligonucleotides will find use, for example, as probes in Southern and Northern blots and as primers in amplification reactions. In preferred embodiments, the oligonucleotides will hybridize specifically to the nucleic acid sequence of SEQ ID NO:1 and identify the alternative splice junction.

Suitable hybridization conditions will be well known to those of skill in the art. In certain applications, for example, substitution of amino acids by site-directed mutagenesis, it is to appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 MM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to MTA1s or, more particularly, homologs of MTA1s from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. RNA Interference

In certain embodiments of the invention may include methods for producing sequence-specific inhibition of gene expression by introducing a double stranded nucleic acid, in particular double-stranded RNA (dsRNA). The process may provide for inhibiting expression of a target gene in a cell. The process comprises introduction of RNA with partial or fully double-stranded character into the cell. Inhibition is typically sequence-specific in that a nucleotide sequence from a portion of the target gene is chosen to produce inhibitory RNA. For exemplary methods related to RNA interference see U.S. Pat. No. 6,506,559; US Patent publications 20030068821, 20030059944, 20030056235, 20030055020, 20030051263, 20020173478, and 20020132788; each of which is incorporated herein by reference.

D. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

E. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

F. Intrabodies

One method of targeting a particular molecule that is expressed at an undesired time or level is the intracellular production of an antibody (intrabody) which is capable of binding to a specific target U.S. Pat. No. 6,004,940, which is incorporated herein by reference. Intrabodies typically do not contain sequences coding for its secretion. These antibodies will bind the target intracellularly. The antibody may be expressed from a DNA sequence which contains a sufficient number of nucleotides coding for the portion of an antibody (intrabody gene) capable of binding to the target. The gene is operably linked to a promoter that will permit expression of the antibody in the cell(s) of interest. Promoters are well known in the art and can readily be selected depending on the cell type desired to be targeted. Furthermore, the use of inducible promoters, which are also well known in the art, in some embodiments are preferred, such as when the function of a target protein is a result of its overexpression.

Cloning the variable region genes for both the V_{H} and V_{L} chains of interest, it is possible to express these proteins in bacteria and rapidly test their function. One method is by using hybridoma mRNA or splenic mRNA as a template for PCR amplification of such genes (Huse, et al., 1989). Thus, one can readily screen an antibody to insure that it has a sufficient binding affinity for the antigen.

G. Aptamers

The methods of the present invention may select and use nucleic acids that bind to and modulate characteristics of MTA1s. Thus, in certain embodiments, a nucleic acid, may comprise or encode an aptamer. An “aptamer” as used herein refers to a nucleic acid that binds a target molecule through interactions or conformations other than those of nucleic acid annealing/hybridization described herein. Methods for making and modifying aptamers, and assaying the binding of an aptamer to a target molecule may be assayed or screened for by any mechanism known to those of skill in the art (see for example, U.S. Pat. Nos. 5,840,867, 5,792,613, 5,780,610, 5,756,291 and 5,582,981, incorporated herein by reference).

IV. Vectors for Cloning, Gene Transfer and Expression

Within certain embodiments expression vectors are employed to express a MTA1s polypeptide product, which can then be purified and, for example, be used to vaccinate animals to generate antisera or a monoclonal antibody with which further studies may be conducted. In other embodiments, MTA1s expression vectors may be used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin | Banerji et al., 1983; Gilles et al., 1983; Grosschedl |
| Heavy Chain | et al., 1985; Atchinson et al., 1986, 1987; Imler |
| | et al., 1987; Weinberger et al., 1984; Kiledjian |
| | et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo |
| | et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-DRa | |
| β-Actin | Kawamoto et al., 1988; Ng et al.; 1989 |
| Muscle Creatine | Jaynes et al., 1988; Horlick et al., 1989; Johnson |
| Kinase (MCK) | et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α₁-Antitrypain | Latimer et al., 1990 |
| H2B (TH2B) | Hwang et al., 1990 |
| Histone | |
| Mouse and/or Type | Ripe et al, 1989 |
| I Collagen | |
| Glucose-Regulated | Chang et al., 1989 |
| Proteins (GRP94 | |
| and GRP78) | |
| Rat Growth | Larsen et al., 1986 |
| Hormone | |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen el at., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987; Glue |
| | et al, 1988 |
| Hepatitis B Virus | Bulla et al., 1986; Jameel et al., 1986; Shaul et al., |
| | 1987; Spandau et al., 1988; Vannice et al., 1988 |
| Human | Muesing et al., 1987; Hauber et al., 1988; |
| Immunodeficiency | Jakobovits et al., 1988; Feng et al., 1988; Takebe |
| Virus | et al., 1988; Rosen et al., 1988; Berkhout et al., |
| | 1989; Laspia et al., 1989; Sharp et al., 1989; |
| | Braddock et al., 1989 |
| Cytomegalovirus | Weber et al., 1984; Boshart et al., 1985; Foecking |
| (CMV) | et al., 1986 |
| Gibbon Ape | Holbrook et al., 1987; Quinn et al, 1989 |
| Leukemia Virus | |

TABLE 3
Inducible Elements

| Element | Inducer | References |
|---|---|---|
| MT II | Phorbol Ester (TFA) | Palmiter et al, 1982; |
| | Heavy metals | Haslinger et al, 1985; |
| | | Searle et al., 1985; Stuart |
| | | et al., 1985; Imagawa |
| | | et al., 1987, Karin et al., |
| | | 1987; Angel et al., |
| | | 1987b; McNeall et al., |
| | | 1989 |
| MMTV (mouse | Glucocorticoids | Huang et al., 1991; Lee |
| mammary tumor | | et al., 1981; Majors et |
| virus) | | al., 1983; Chandler et |
| | | al., 1983; Lee et al., |
| | | 1984; Ponta et al., 1985; |
| | | Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV4O | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large T | Taylor et al., 1989, |
| | Antigen | 1990a, 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al., 1994; Kelly et al., 1995), the α actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the creatine kinase promoter (Ritchie, 1996), the α7 integrin promoter (Ziober and Kramer, 1996), the brain natriuretic peptide promoter (LaPointe et al., 1996), the α B-crystallin/small heat shock protein promoter (Gopal-Srivastava, 1995), and α myosin heavy chain promoter (Yamauchi-Takihara et al., 1989) and the ANF promoter (LaPointe et al., 1996).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Constructs

There are a number of ways in which expression constructs may be introduced into cells. In certain embodiments of the invention, a vector (also referred to herein as a gene delivery vector) is employed to deliver the expression construct. By way of illustration, in some embodiments, the vector comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Where viral vectors are employed to deliver the gene or genes of interest, it is generally preferred that they be replication-defective, for example as known to those of skill in the art and as described further herein below.

One of the preferred methods for in vivo delivery of expression constructs involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized.

In preferred embodiments, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage and are able to infect non-dividing cells such as, for example, cardiomyocytes. So far, adenoviral infection appears to be linked only to mild disease such as acute respiratory disease in humans.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of such adenovirus vectors is about 7.5 kb, or about 15% of the total length of the vector. Additionally, modified adenoviral vectors are now available which have an even greater capacity to carry foreign DNA.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector.

As stated above, a preferred adenoviral vector according to the present invention lacks an adenovirus E1 region and thus, is replication. Typically, it is most convenient to introduce the polynucleotide encoding the gene of interest at the position from which the E1-coding sequences have been removed. However, the position of insertion of the construct within the adenovirus sequences is not critical to the invention. Further, other adenoviral sequences may be deleted and/or inactivated in addition to or in lieu of the El region. For example, the E2 and E4 regions are both necessary for adenoviral replication and thus may be modified to render an adenovirus vector replication-defective, in which case a helper cell line or helper virus complex may employed to provide such deleted/inactivated genes in trans. The polynucleotide encoding the gene of interest may alternatively be inserted in lieu of a deleted E3 region such as in E3 replacement vectors as described by Karlsson et al. (1986), or in a deleted E4 region where a helper cell line or helper virus complements the E4 defect. Other modifications are known to those of skill in the art and are likewise contemplated herein.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include administration via intracoronary catheter into one or more coronary arteries of the heart (Hammond, et al., U.S. Pat. Nos. 5,792,453 and 6,100,242) trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results-in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. In general, viral vectors accomplish delivery of the expression construct by infecting the target cells of interest. Alternatively to incorporating the expression construct into the genome of a viral vector, the expression construct may be encapsidated in the infectious viral particle.

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression vector may simply consist of naked recombinant DNA or plasmids comprising the expression construct. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in viva (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

V. Diagnosing and Treating Overexpression of MTA1s

The inventors believe that MTA1s plays an important role in the development of hormone insensitive cells or disease and, further, in the mechanisms of hormone independent and/or metastatic cancers. Thus, in another embodiment, there are provided methods for diagnosing defects in MTA1s expression and function. More specifically, regulatory pertubations relating to MTA1s, as well as increases or decreases in levels of expression, may be assessed using standard technologies, as described below. Disease may include, but are not limited to osteoporosis, osteoarthritis, osteosarcoma, coronary heart disease (CHD), coronary artery disease (CAD), atherosclerosis, Alzheimer's disease, abnormal uterine bleeding, early menarche, fibrocystic breast disease, fibroids of the uterus, late menopause, ovarian cysts, PMS, various skin disorders, as well as cancer of the breast, uterus, ovary, kidney, testis, prostate, skin, intestine, stomach, liver and other tissues.

A. Genetic Diagnosis of MTA1s Alterations

One embodiment of the instant invention comprises a method for detecting variation in the expression of MTA1s. This may comprise determining the level of MTA1s or determining specific alterations in the expressed product.

A suitable biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow, kidney or cells or tissue derived from these tissues or organs. Other embodiments include fluid samples such as peripheral blood, lymph fluid, ascites, serous fluid, pleural effusion, sputum, cerebrospinal fluid, lacrimal fluid, stool or urine.

The nucleic acids used for diagnosis are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. The nucleic acid may also be amplified using known nucleic amplification prodedures.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including overexpression, deletions, insertions, point mutations and duplications. Point mutations result in stop codons, frameshift mutations or amino acid substitutions. Somatic mutations are those occurring in non-germline tissues. Germ-line mutations are typically inherited. Mutations in and outside the coding region also may affect the amount of MTA1s produced, both by altering the transcription of the gene or in destabilizing, stabilizing or otherwise altering the processing of either the transcript (mRNA) or protein.

It is contemplated that other mutations in the MTA1s genes may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

1. Primers and Probes

The term primer, as defined herein, is meant to encompass any nucleic acid that is capable of priming the synthesis of a nascent nucleic acid that spans the splice junction producing MTA1s in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

2. Template Dependent Amplification Methods

A number of template dependent processes are available to amplify the marker sequences present in a given template sample to identify the presence of the splice variant that results in the MTA1s protein. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence and in various embodiments of the invention with in a particular number of nucleotides from the splice junction. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polyrmerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

3. Southern/RNA Blotting

Blotting techniques are well known to those of skill in the art. Southern blotting involves the use of DNA as a target, whereas RNA blotting (northern blotting) involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

4. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

5. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the MTA1S genes that may then be analyzed by direct sequencing.

6. Kit Components

All the essential materials and reagents required for detecting and sequencing MTA1s and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

B. Immunologic Diagnosis

Antibodies of the present invention can be used in characterizing the MTA1s content of healthy and diseased tissues, through techniques such as ELISAs, immunohistochemistry and Western blotting. These methods may provide a screen for the presence or absence of metastatic cancer or hormone insensitive cancer, as well as a predictor of disease prognosis.

The use of antibodies of the present invention, in an ELISA assay is contemplated. For example, anti-MTA1s antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for MTA1s that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

VI. Treating Defects MTA1s Expression of Function

The present invention also involves, in another embodiment, the treatment of disease states related to the aberrant expression and/or function of MTA1s. In particular, it is envisioned that MTA1s activity plays a role in development of hormone insensitive cancers. Thus, decreasing levels of MTA1s, or reducing or eliminating the activity of MTA1s, are believed to provide therapeutic intervention in certain cancers.

There also may be situations where one would want to inhibit MTA1s function or activity, for example, where overexpression or unregulated expression had resulted in insensitivity to hormones or inappropriate modulation of non-genomic pathways. In this case, one would take steps to interfere with or block the expression or function of MTA1s.

A. Genetic Based Therapies

One of the therapeutic embodiments contemplated by the present inventors is the intervention, at the molecular level, in the events involved in some cancers. Specifically, the present inventors intend to provide, to a hormone insensitive cell, a negative modulator capable of decreasing or inhibiting MTA1s in that cell. In certain embodiments, the negative modulator of MTA1s may be an expression construct providing an anti-MTA1s molecule, such as an anti-sense nucleic acid. The lengthy discussion of expression vectors and the genetic elements, as well as anti-sense methodology employed therein is incorporated into this section by reference. Particularly preferred anti-sense expression vectors are viral vectors such as adenovirus, adeno-associated virus, herpesvirus, vaccinia virus and retrovirus. Also preferred are liposomally-encapsulated expression vectors.

Those of skill in the art are aware of how to apply gene delivery to in vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below. Various routes are contemplated, including local and systemic, but targeted provision to the cancer of interest is preferred. (See, for example Hammond, et al., 1994, hereby incorporated by reference in its entirety.)

Also contemplated is the use of RNA interference to modulate the expression of MTA1s in a cell. Related methods involve the use of double-stranded RNA (“dsRNA”) or RNA that forms a double stranded structure, such as a hairpin or other self complementary structure, that are sufficiently homologous to a portion of the MTA1s gene product such that the dsRNA degrades mRNA that would otherwise affect the production of MTA1s. A well-defined 21-base duplex RNA, referred to as small interfering RNA (“siRNA”), may operate in conjunction with various cellular components to silence the MTA1s gene product with sequence homology. For exemplary methods see U.S. Pat. No. 6,506,559, and the publications of Hammond et al., Nature 110-119 (2001); Sharp, Genes Dev. 15:485490 (2001); and Elbashir, et al., Genes Dev. 15:188-200, each of which is incorporated by reference herein in its entirety.

B. Combined Therapy

In many clinical situations, it is advisable to use a combination of distinct therapies. Thus, it is envisioned that, in addition to the therapies described above, one would also wish to provide to the patient more “standard” pharmaceutical cancer therapies. Examples of standard therapies include radiation, gene therapy, or chemotherapeutics.

Combinations may be achieved by contacting cancer cells with a single composition or pharmacological formulation that includes both agents, or by contacting the cell with two distinct compositions or formulations, at the same time, wherein one composition may include a MTA1s expression construct, therapeutic peptides or other therapies, and the other includes the standard cancer therapy agent. Alternatively, a MTA1s therapeutic may precede or follow the other treatment by intervals ranging from minutes to weeks. In embodiments where the other agent and MTA1s therapeutic are applied separately to the cell, one would generally ensure that a significant period of time did not expire between the time of each delivery, such that the agent and MTA1s therapeutic would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a negative modulator of MTA1s nucleic acid or protein, or the other agent will be desired. Various combinations may be employed, where a negative modulator of MTA1s is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated as well.

C. Formulations and Routes for Administration to Patients

Where clinical applications are contemplated, it will be necessary to prepare pharmaceutical compositions—expression vectors, virus stocks, aptamers, peptides and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal, intravascular or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a maimer compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

VII. MTA1S Expressing Cell Lines

A particular embodiment of the present invention provides host cells that contain MTA1s-related constructs. Host cells expressing MTA1s, including recombinant cell lines derived from such host cells, may be useful in methods for screening for and identifying agents that modulate a function or activity of MTA1s, and thereby alleviate pathology related to the over expression of these molecules. The use of constitutively expressed MTA1s provides a model for over-regulated expression.

In a general aspect, a host cell is produced by the integration of a MTA1s transgene into the genome in a manner that permits the expression of MTA1s.

VIII. Screening Assays

The present invention also contemplates the screening of compounds for various abilities to interact and/or affect MTA1s expression or function. Particularly preferred compounds will be those useful in inhibiting the actions of MTA1s in pre-cancerous or cancerous cells, tissues, or organs. In the screening assays of the present invention, the candidate substance may first be screened for basic biochemical activity—e.g., binding to MTA1s—and then tested for its ability to modulate activity or expression, at the cellular, tissue or whole animal level.

A. Assay Formats

The present invention provides methods of screening for modulators of MTA1s. In one embodiment, the present invention is directed to a method of:

(i) providing a MTA1s polypeptide;

(ii) contacting the MTA1s polypeptide with the candidate substance; and

(iii) determining the binding of the candidate substance to the MTA1s polypeptide.

In yet another embodiment, the assay looks not at binding, but at MTA1s expression. Such methods would comprise, for example:

(i) providing a cell that expresses MTA1s polypeptide;

(ii) contacting the cell with the candidate substance; and

(iii) determining the effect of the candidate substance on expression of MTA1s.

In still yet other embodiments, one would look at the effect of a candidate substance on the activity of MTA1s. This may involve looking at any of a number of cellular characteristics, including Steroid Hormone responsive genes or expression of a reporter gene. Of particular interest will be measuring steroid hormone receptor activity, such as sensitivity to estrogen and activity of the estrogen receptor. An exemplary assay may include a reporter plasmid with a palindromic ERE, derived from vitellogenin A2 promoter or other estrogen responsive gene, and CAT gene or other reporter gene driven by a partial promoter sequence of thymidine kinase (Pietras et al., 1995; Ernst et al., 1991). MCF-7 breast cancer cells may be used to establish transient transfection assays that allow assay of ERE-dependent induction of CAT activity. CAT protein is assessed by established methods (Pietras et al., 1995). Candidate substances such as small molecules or peptides are delivered in solution or by use of liposomes (phosphatidylcholine, stearylamine and cholesterol) using methods described in detail before (Pietras, 1978; Magee et al., 1974). Other methods for assaying substances for the ability to dissociate MTA1s and proteins that bind MTA1s are known in the art, for examples see U.S. Pat. Nos. 5,445,941 and 6,365,361, which are herein incorporated by reference.

Various reporter gene assays may be used to assay substances that modulate the interaction between MTA1s and other proteins that bind with MTA1s. A reporter gene is a coding sequence attached to heterologous promoter and/or enhancer elements and whose product is easily and quantifiably assayed when a reporter gene construct is introduced into tissues or cells. Exemplary reporter genes include β-galactosidase (lacZ), chloramphenicol acetyltransferase (cat) and β-glucuronidase (gus), as well as other known in the art. These reporter genes may be positioned under the control of a promoter or enhancer that is directly or indirectly modulated by protein that interacts with MTA1s. The modulation of the interaction may lead to the disruption or maintenance of protein-protein interaction resulting in the activation or inhibition of expression of the reporter. The expression of the reporter gene may then be quantitated and compared to controls to determine the effect of the substance in modulating MTA1s activity or interaction affinity for other proteins of interest.

Assays may be performed in vitro or in vivo (e.g., in mammalian, yeast, or bacterial cells). Expression constructs may contain all or part of the polynucleotides involved in binding or expression of the reporter construct. Fusion proteins DNA binding or transactivation domains may also be incorporated, such as those used in the yeast two hybrid screens.

B. Inhibitors and Activators

An inhibitor according to the present invention may be one which exerts an inhibitory effect on the expression or function/activity of MTA1s. By the same token, an activator according to the present invention may be one which exerts a stimulatory effect on the expression or function/activity of MTA1s.

C. Candidate Substances

As used herein, the term “candidate substance” refers to any molecule that may potentially modulate MTA1s expression or function. The candidate substance may be a protein or fragment thereof, a small molecule inhibitor, or even a nucleic acid molecule. It may prove to be the case that the most useful pharmacological compounds will be compounds that are structurally related to compounds which interact naturally with MTA1s. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like a MTA1s, and then design a molecule for its ability to interact with MTA1s. Alternatively, one could design a partially functional fragment of a MTA1s (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of a steroid hormone receptor repressor.

Other suitable inhibitors include antisense molecules, ribozymes, aptamers, intrabodies, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

D. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a MTA1s molecule or fragment thereof is provided.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as a MTA1s). Competitive binding assays can be performed in which one of the agents (MTA1s for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a MTA1s and washed. Bound polypeptide is detected by various methods.

Purified target, such as a MTA1s, can be coated directly onto plates for use in the aforementioned drug screening techniques. However, non-neutralizing antibodies to the polypeptide can be used to immobilize the polypeptide to a solid phase.

E. In cyto Assays

Various cell lines that express MTA1s can be utilized for screening of candidate substances. For example, cells containing a MTA1s with engineered indicators can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (reporter gene expression). Alternatively, molecular analysis may be performed in which the function of a MTA1s and related pathways may be explored. This involves assays such as those for protein expression, enzyme function, substrate utilization, mRNA expression (including differential display of whole cell or polyA RNA) and others.

F. Production of Inhibitors

In an extension of any of the previously described screening assays, the present invention also provide for method of producing inhibitors. The methods comprising any of the preceding screening steps followed by an additional step of “producing the candidate substance identified as a negative modulator of” the screened activity.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods

Cell Cultures and Antibody Development.

Breast Cancer MCF-7 and ZR-75R cells '4 were maintained in Dulbecco's modified Eagle's medium (DMEM)-F 12 (1:1) supplemented with 10% fetal calf serum. ZR-75R cells were supplemented with I mM sodium pyruvate, and 0.5 μg hydrocortisone/ml. The 20-amino-acid peptide was cross-linked to the keyhole cyanate vector protein and used to immunize New Zealand white rabbits at Research Genetics, Inc. Huntsville, Ala.

Cloning.

A 290-bp MTA-1 DNA fragment amplified by RT-PCR from the MCF-7 cells was used as a probe to isolate the MTA1 cDNAs from a human mammary gland library (Promega, California). The MTA-1 primer sequences were as follows: SEQ ID NO: 5 forward, 5′-AGCTACGAGGAGGACAACGGG-3′; SEQ ID NO: 6 reverse, 5′-CACGCTTGGTTTCCGAGGAT-3′. MTA1 clones were subcloned into the T7-pcDNA and GST vector. Unique primer sequences to selectively amplify full-length MTA1s cDNAs were: forward primer of SEQ ID NO: 14 was 5′-ATGCCGTCAACARGTACAGGGTCGGAGACTAC-3′ and reverse primer of SEQ ID NO: 15 was 5′-TGGGCTCTCTCCATCTAACCG-3′

Transfection and Promoter-Reporter Assay.

Transfection was performed using a Fugene-6 kit (Roche Biochemical, New Jersey) as per the manufacturer's instructions. Cells were transiently cotransfected with a reporter construct and (β-galactosidase, and assayed for luciferase activity as described.

In Vitro Transcription and Translation.

In vitro transcription and translation of the MTA1, protein was performed using the TNT-transcription-translation system. One 1 μg of MTA1 cDNAs in pcDNA 3.1 vector was translated in the presence of ³⁵S-methionine using a T7-TNT kit as described.

GST Pull-Down Assay.

Incubating equal amounts of GST performed the GST pull-down assays or GST-AF2 or GST-AF 1 proteins immobilized to glutathione-sepharose beads with in vitro translated ³⁵S-labelled MTA1s, protein as described.

RT-PCR and RNase Protection Assay.

RT-PCR was performed by using the access RT-PCR system (Promega) as per manufacturer's instructions. The primers used for amplifying the MTA1 +1023 to +1355 region were as follows, SEQ ID NO: 7 forward, 5′-AAGACCACCGACAGATACGTGC-3′; SEQ ID NO: 8 reverse, 5′-TGGCCTCTCTCCATCTAACCG-3′. The amplified fragments were cloned into Topo II vector (Invitrogen). The PCR fragments were cloned into Topo II vector (Invitrogen). Riboprobes were synthesized using in vitro transcription assay system (Promega) and labeled with ³²P per instructions from the manufacturer. Probes used in RNase protection assays included MTA1 probe of SEQ ID NO: 12 and an MTA1s probe of SEQ ID NO: 13.

Two-Hybrid Screening.

A yeast two-hybrid based assay was performed using the Matchmaker yeast two-hybrid system-3 in accordance with the manufacturer's protocol (Clontech, California). To construct GBD-MTA1s-full, we subcloned a blunt-ended BamHI—XbaI fragment containing the open reading frame of MTA1s, into the BamHI and Pst1 (blunt end) sites of the GAL4 binding domain vector pGBKT7. GAD-ERα was constructed by subcloning the ERα open reading frame using BamnHI and EcoRI sites from CMV5-ERα15 into GAL4 activation domain containing vector pGADT7.

Yeast AH109 strain was used since it has three distinct selection genes (-Ade, -His and β-Gal reporters), all of which interact with GAL4 transcription factor. In this assay, yeast cells were transformed with the MTA1s plasmid as a fusion of GAL4 binding domain and ER fusion with GAL4 activation domain. The interaction of MTA1s and ER reconstitutes functional GAL4 protein, which enables yeast cells to grow on plates lacking adenine, histidine, leucine, and tryptophan (Ade- His- Leu-Trp). GAD-ERα amino acids 1-536 or GBD-MTA1s-amino acids 1-429 alone failed to support the growth of yeast on these plates. However, the yeast did grow on plates in the presence of both ER and MTA1s but not with N-ter or C-ter deletions of MTA1s. In addition, the interaction of MTA1s and ER activated β-Gal as demonstrated by blue colonies. Further, the interaction between ER and MTA1s was not influenced by estradiol treatment. These results suggested that full-length MTA1s is required for the functional interaction of MTA1s and ER, confirming the interaction of MTA1s with ER.

Production of Stable Cell Lines Expressing MTA1s.

MCF-7 and ZR-75R cells were transfected with pcDNAT7-MTA1s by using calcium phosphate method. The positive clones expressing a stable level exogenous MTA1s, were identified by immunoblotting using anti-T7 mAb.

Immunofluorescence Confocal Assays.

The cellular localization of T7-MTA1s, and ER was determined using indirect immunofluorescence as described in Mazumdar et al., 2001, herein incorporated by reference. Cells were treated with or without anti-T7 mAb or ER Ab followed by 546-Alexa-labeled goat anti-mouse Ab or 488-Alexa.

Construction of MTA-1s Mutants.

Mutation of MTA1s was performed by using the Quick-change kit (Stratagene) per manufacturer's instructions. The following primers were used generate a mutant lacking LRILL, SEQ ID NO: 9 forward, 5′-GAGAGCTGTTACATGTCGTCTGACATATTGGAAGAAATATGGTGG; SEQ ID NO: 16 Reverse, Del R5′-CCACCATATTTCTTCCAATATGTCA-GACGACATGTAACAGCTCTC. The following primers were used to add a stop codon to generate a mutant lacking the 33 novel amino acids, SEQ ID NO: 17 forward, 5′-GGGCCTGCGAGAGCTGTTAATGTCGTCTCTGCGCATCTTGTTG; SEQ ID NO: 18 reverse, 5′-CAACAAGATGCGGAGAGACGACATTAACAGCTCTCGCAGGCCC.

Soft-Agar Growth and Tumorigenesis Assays.

Soft agar colony growth assays were performed as described in Mazumdar et al., 2001, herein incorporated by reference. For xenografts studies, 5×10⁶ cells were implanted subcutaneously into mammary fat-pad of nude mice and allowed to grow for 21 days and tumor size was measured, as described in Mandal et al., 2001, herein incorporated by reference.

Human Tissue Samples.

Residual specimens derived from patients who had undergone routine surgery for breast cancer were snap frozen in liquid nitrogen and stored at −80° C., as described in Al Saati et al., 1993, herein incorporated by reference. ER status was determined as a part of routine diagnostic pathology. Estrogen receptor status of the primary tumors were determined by immunohistochemical staining using mouse monoclonal antibody ID5 (Zymed Laboratories) directed against estrogen receptor (Mandal et al., 2001). The ER positivity was determined by assessing the percentage of the nuclear ER in the invasive carcinoma component of the tumors. Tumors showing positive staining in more than 10% of the tumor nuclei were considered as positive. Results shown are representative of 3 to 5 separate experiments with similar findings.

### Example 2

### Identification of MTA1s Variant

The MTA1 complementary DNAs (cDNA) of varying lengths from a human mammary gland cDNA library were cloned. The MTA1 cDNA were sequenced and subcloned into pcDNA3.1 expression vector using MTA1 cDNAs open reading frames (FIG. 1A). To investigate the functionality of these clones, MTA1 cDNAs were translated in vitro and resolved the resulted protein products onto SDS-polyacrylamide gels (FIG. 1B). All of the MTA1 clones except the S2 (MTA1s) clone were translated into proteins of expected sizes. The MTA1s clone was translated into a protein of 44 kDa rather than the 70-kDa expected size, based on the MTA1s cDNA length. To confirm the translation of the MTA1s gene in vivo, MCF-7 breast cancer cells were transiently transfected with a T7-tagged MTA1S cDNA, and the protein extracts were analyzed by western blotting with an anti-T7 antibody. The MTA1s clone generated a 54-kDa protein, compared with the 80-kDa protein from the full-length MTA1-T11 (FIG. 1C).

Since MTA1 is a corepressor of ER (Mazumdar, et al., 2001, incorporated herein by reference), the ability of MTA1 cDNAs to repress the transactivating function of ER were investigated. The transient expression of MTA1s or full-length MTA1 but not other MTA1 cDNAs effectively blocked estradiol's ability to stimulate ERE transcription (FIG. 1D). In addition, MTA1s clone also blocked the stimulation of the glucocortocoid responsive element (GRE) transcription by dexamethasone (FIG. 1E) and progesterone responsive element (PRE) transcription by progesterone, but not retinoic-acid responsive element (RARE) transcription (FIG. 1F).

A thorough analysis of the MTA1s sequence revealed a deletion of 47 bases between the +1224 and +1272 of MTA1 (Genebank accession U35113), and was generated by alternative splicing (FIG. 1G). This deletion caused a reading frame-shift, leading to an addition of a unique 33 amino acid sequence, which had no significant homology with sequences in the protein database, except for the presence of a potential steroid receptor binding motif LRILL (FIG. 1H). Comparison of sequences at the deletion site revealed that 3′ donor site has a consensus site for splicing (Shapiro and Senapathy, 1987, incorporated herein by reference) while 5′ has a non-consensus splicing acceptor site. Similar functional non-consensus splicing site has been reported in cell cycle PISTLRE gene (Crawford, et al.,1999).

Because this new MTA1 variant was much shorter than MTA1, it was named the MTA1s (short) or MTA1s (clone isolate). Since MTA1 and MTA1s differ from each other by 47 bp, natural transcripts encoding MTA1 and MTA1s could not be sufficiently resolve by Northern blotting. Thus, Southern blot analysis after a reverse transcription polymerase chain reaction, amplifying the region spanning from +1023 to +1355 of the MTA1 with the RNA extracted from MDA-MB435 breast cancer cells were performed. As expected, two distinct bands appeared, corresponding to those generated from MTA1 and MTA1s, plasmids, respectively (FIG. 1I). The lower DNA band was sequenced and was identical to that of MTA1s. To further confirm the expression of MTA1s in cancer cells, RNAse protection assays were used. Since MTA1s has a deletion of 47 base pair, binding of MTA1 probe to MTA1 transcript is expected to protect a band of 332 bp and binding of MTA1s, probe will generate two fragments of 201 and 84 bp sizes due to cleavage by RNAse. As expected, MTA1specific probe protected a 332 bp band and also 201 and 84 bp bands (FIG. 1J, lanes 3, 4)]. While in another experiment, a MTA1s probe protected a 285-bp MTA1s specific fragment and MTA1 was cleaved into 201 and 84 bp bands (lane 6). Together, these results showed the natural existence of MTA1s, transcripts in breast cancer cell lines.

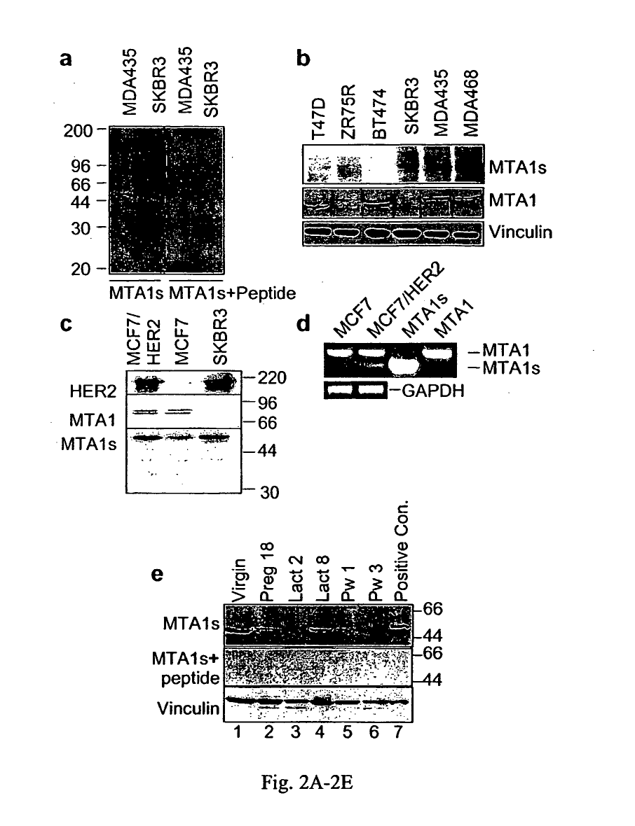

A polyclonal antibody against the last 20 amino acids of MTA1s was produced and showed that the antibody specifically recognized the GST-MTA1s protein and an endogenous 55-kDa MTA1s protein band in MCF-7, T47D, and MDA-MB231 cells (FIG. 2A). In the mammary glands, the MTA1s, protein expression and electrophoretic motility were differentially regulated as a function of the developmental stage, suggesting that the MTA1s protein be modified during mammary gland development (FIG. 2B). Elevated levels of MTA1s protein were expressed in the brain, testes, ovaries, adrenal glands, and virgin mammary glands. In contrast, MTA1s protein levels were extremely low in the salivary and pituitary glands (FIG. 2C). The expression profile of MTA1s was quantitatively distinct from that of MTA1. For example, MTA1 was clearly expressed in the pituitary gland whereas MTA1s was barely detectable. In contrast, MTA1s expression in the adrenal glands was elevated whereas the expression level of MTA1 was low (FIG. 2C). The differential regulation of these two protein products from the same gene suggests that the function of MTA1s may be different from that of MTA1.

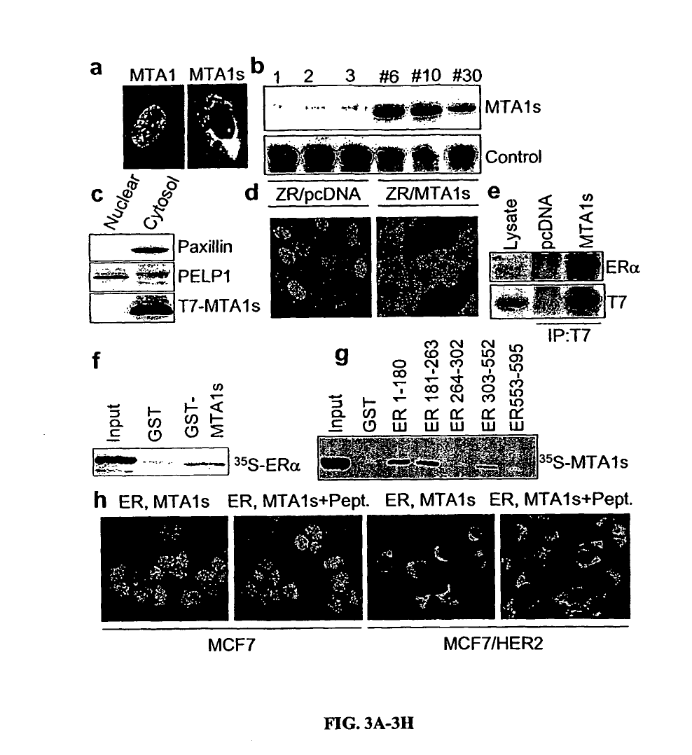

Since MTA1s lacked the nuclear signal sequence, the subcellular localization and function of MTA1s was examined. Confocal microscopy using an anti-T7 mAb showed that the T7-tagged MTA1s was localized in the cytoplasm whereas MTA1 was in the nucleus as expected (FIG. 3A). The role of MTA1s, in breast cancer cells, was investigated by generating stable clones of ZR-75R cells expressing T7-MTA1s (FIG. 3B). Subcellular fractionation of the ZR-75R cells expressing MTA1s confirmed that T7-MTA1s was in the cytoplasm. To assess the purity of the fractions, immunoblotting with antibodies against the nuclear corepressor PELP1 (Vadlamudi, et al., 2000) and cytosolic paxillin (FIG. 3C) were used. To understand the impact of MTA1s on the status of ER, immunostaining with an anti-ER mAb were used on MCF-7 cells that were transfected with the T7-MTA1s. The expression of T7-MTA1s was accompanied by a significant reduction in the nuclear ER immunoreactivity (FIG. 3D), raising the possibility that MTA1s regulates ER. To confirm the interaction between the MTA1s and endogenous ER, immunoprecipitation of T7-MTA1s with an anti-T7 mAb was used. The results showed that the T7-MTA1s protein physically associated with ER (FIG. 3E). To investigate whether the association between MTA1s and ER was direct or mediated by other proteins, the ability of in vitro-translated ER protein to bind with GST-MTA1s was investigated. MTA1s interacted with the GST-ER but not with GST alone in GST pull-down assays (FIG. 3F). In a reverse experiment, the binding ability of in vitro translated MTA1s with the GST-AF2 domain of the ER was examined (Kumar, et al., 1987 and Simoncini et al., 2000). MTA1s interacted with the ER but not to GST alone (FIG. 3G).

A yeast-based two-hybrid assay to show the potential interaction of ERα and MTA1s in vivo was used (FIG. 3H-J). The Yeast AH109 strain was used since it has three distinct selection genes (-Ade, -His and β-Gal reporters), all of which interact with GAL4 transcription factor. In this assay, yeast cells were transformed with MTA1s plasmid as a fusion of GAL4 binding domain and ERα was expressed as a fusion of GAL4 activation domain. The interaction of MTA1s and ERα reconstitutes functional GAL4 protein, which enables yeast cells to grow on plates lacking adenine, histidine, leucine, and tryptophan (Ade- His- Leu-Trp). GAD-ERα or GBD-MTA1s-full alone failed to support the growth of yeast on these plates (FIG. 3J). However, the yeast did grow on plates in the presence of both ERα and MTA1s but not with amino terminal or carboxy terminal deletions of MTA1s. In addition, the interaction of MTA1s and ERα activated β-Gal as demonstrated by the blue color of the colonies (FIG. 3J, last panel). Further, the interaction between ERα and MTA1s was not dependent on estradiol, and antiestrogen ICI 182780 failed to block this interaction. These results suggested that full-length MTA1s is required for the functional interaction of MTA1s and ERα and hence confirmed the interaction of MTA1s with ERα.

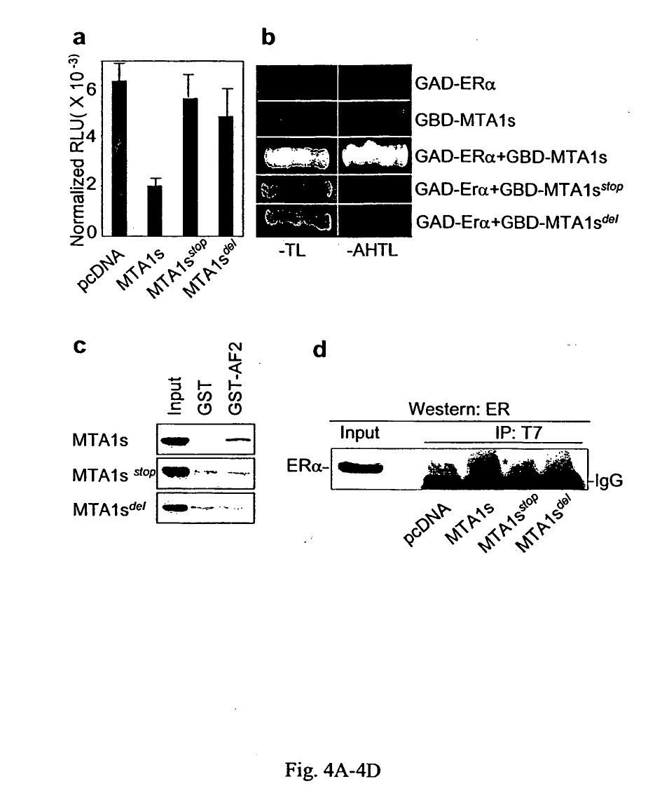

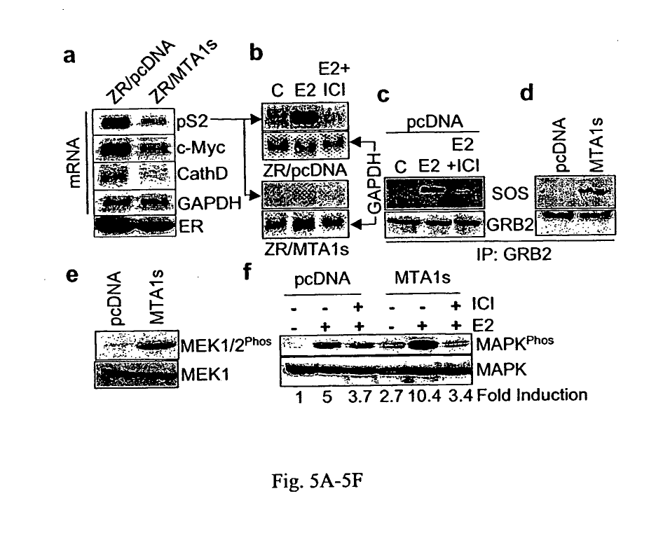

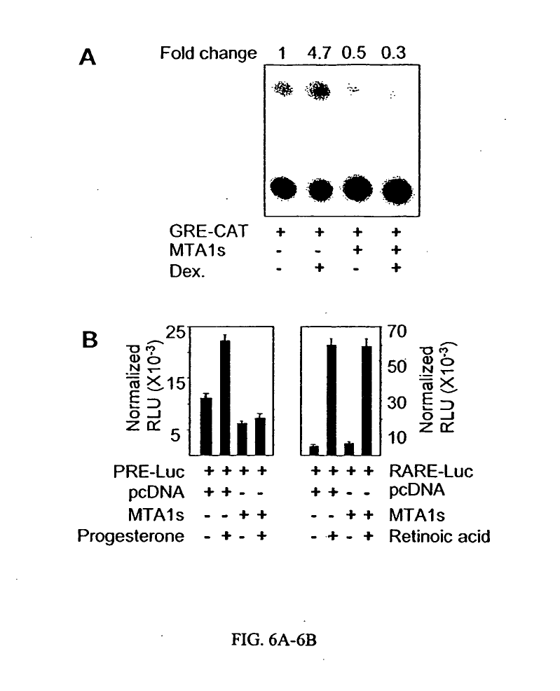

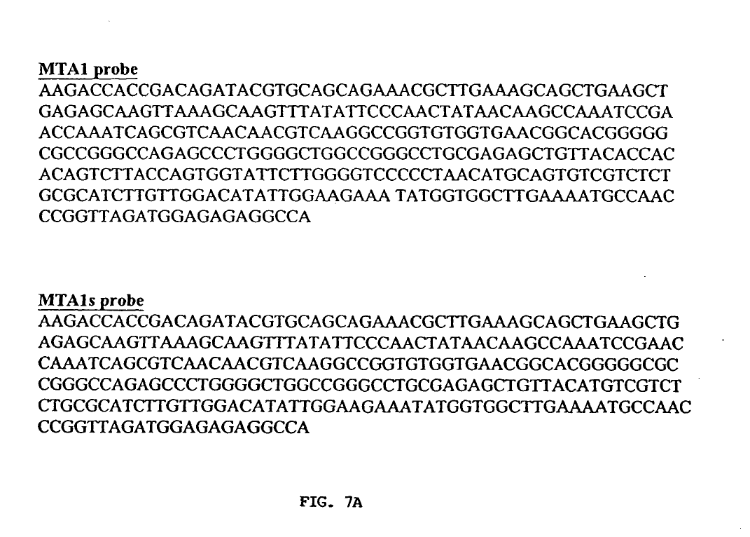

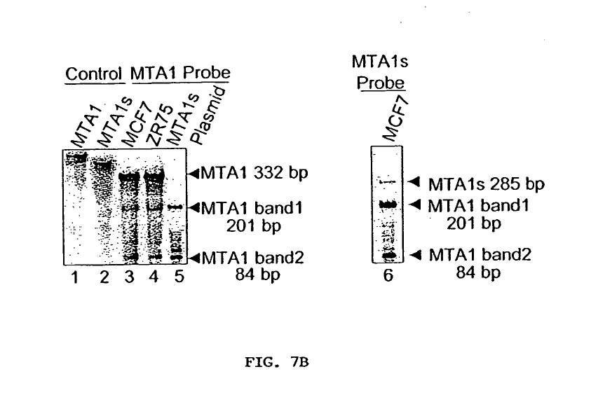

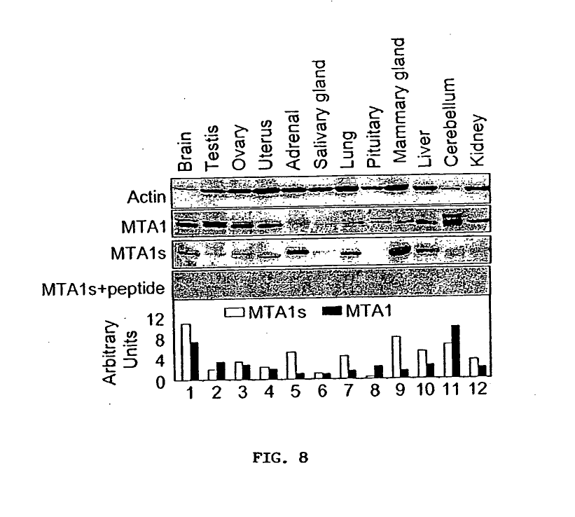

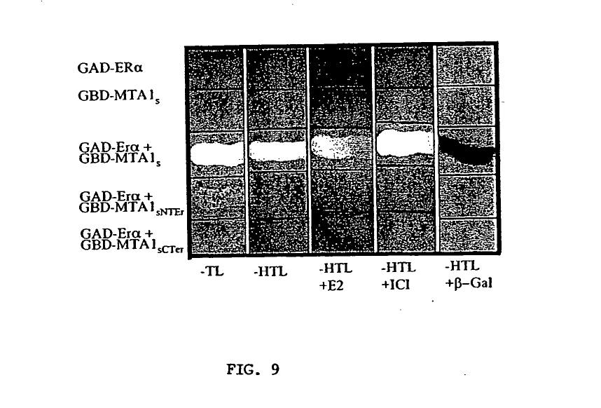

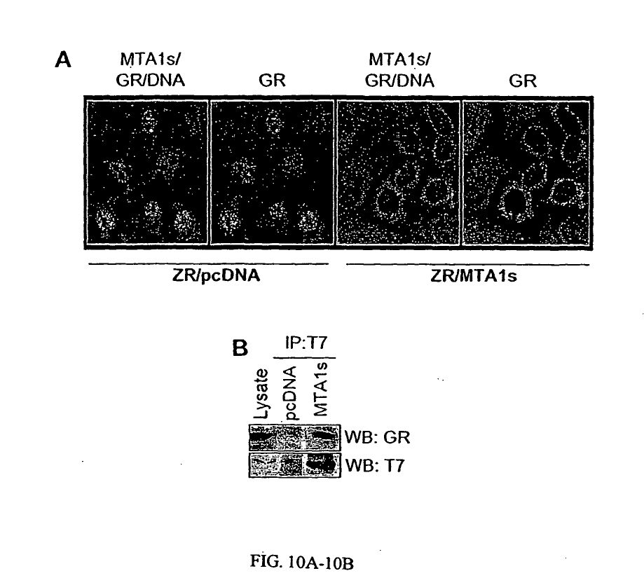

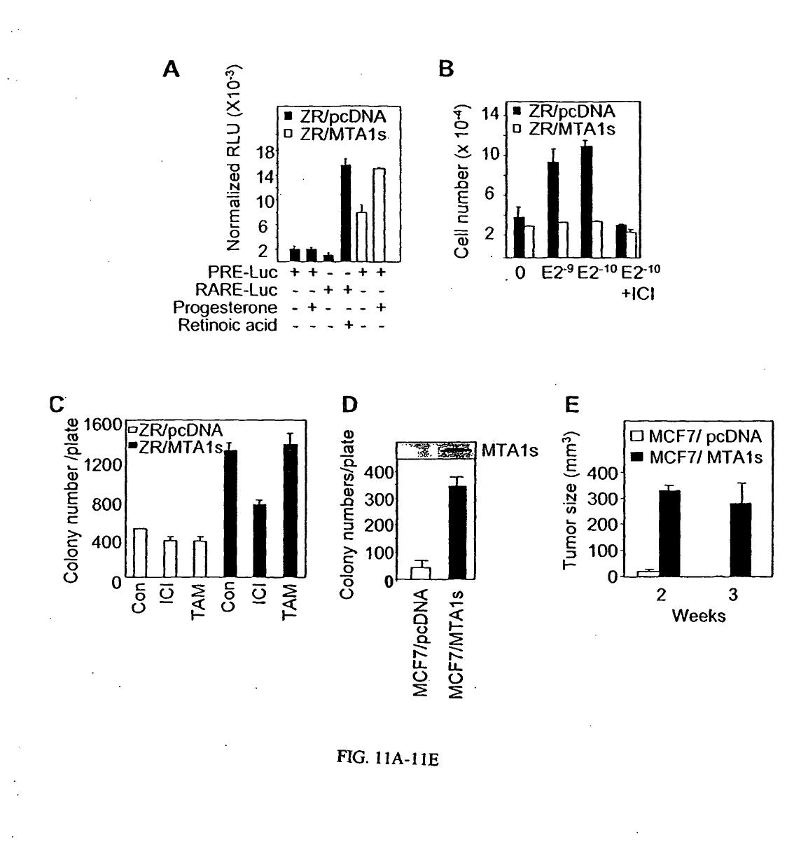

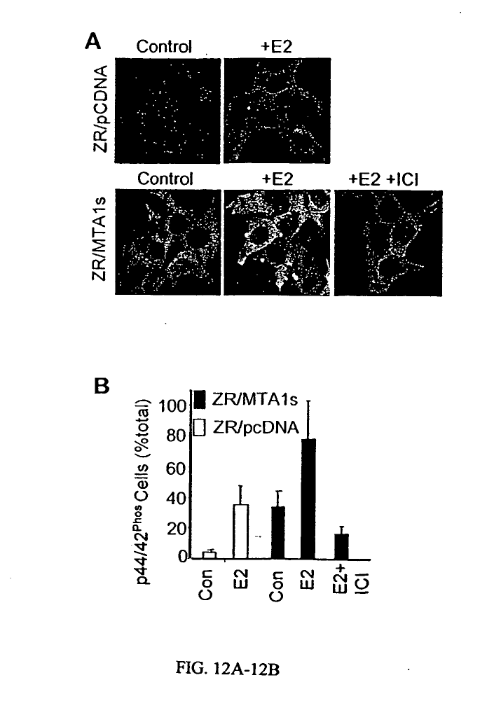

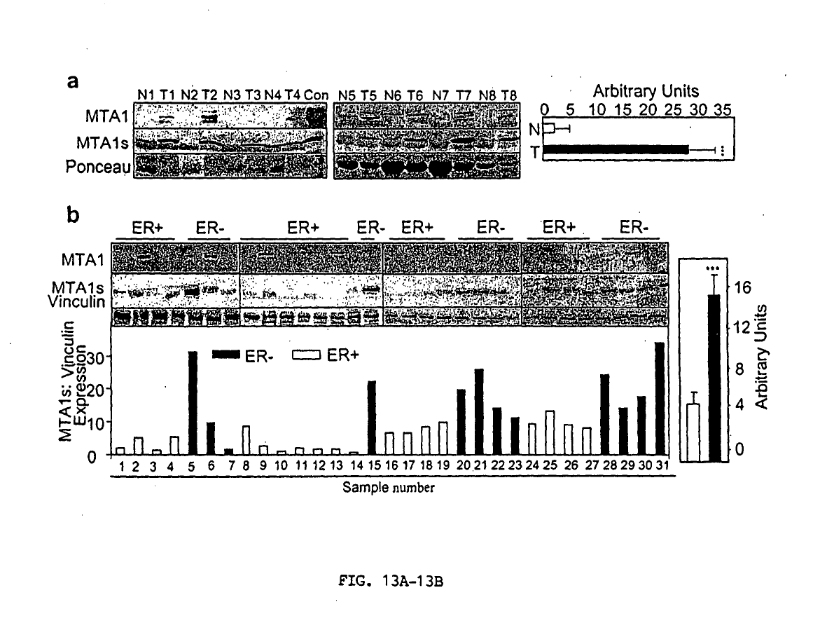

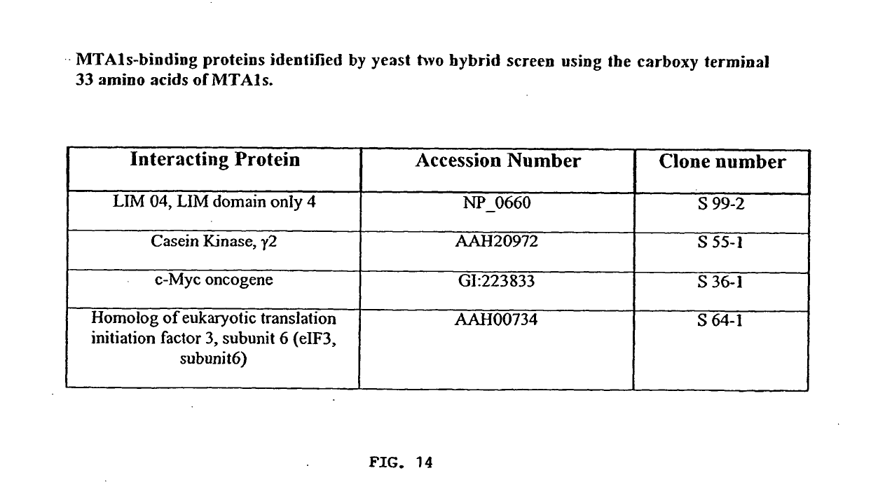

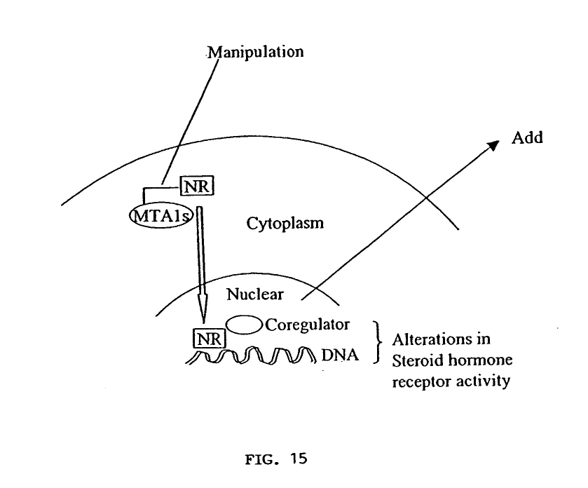

To demonstrate that MTA1s is necessary for ERα retention in breast cancer cells, the biochemical basis of MTA1s retention of ERα in the cytoplasm was determined. Since the novel sequence of 33 amino acids contains a potential nuclear receptor binding site LRILL (FIG. 1H), the significance of 33 amino acids and LRILL were investigated by creating two MTA1s mutants, one by adding a stop codon and deleting the entire 33 amino acids (named MTA1s-stop, and a second one by deleting the LRILL motif (named MTA1s-del). As oppose to the wild type MTA1s, transient expression of MTA1s-stop and MTA1s-del did not result in a significant suppression of estradiol's ability to stimulate ERE transcription (FIG. 4A). Deletion of LRILL or novel 33 amino acids also prevented the ability of MTA1s to interact with the ERα in two-hybrid assay (FIG. 4B), with the AF2 domain of ERα in the GST-pull down assay (FIG. 4C), and with the endogenous ERα in MCF-7 cells (FIG. 4D). These observations suggested the requirement of the putative steroid binding motif LRILL to mediate the observed interaction of MTA1s with ERα. To assess the role of LRILL motif in the noticed sequestration of ERα in the cytoplasm, it was determined whether deletion of LRILL will release the ERα to the nucleus in estradiol stimulated MCF-7 cells. Expression of MTA1s-del but not wild type MTA1s was accompanied by the presence of ERα in the nucleus in more than 90% of the cell expressing MTA1s-del. Together, these findings indicated a pivotal role of LRILL motif in the cytoplasmic sequestration of ERα.

The effect of estradiol on MTA1s interaction with the ER in ZR-75R cells expressing MTA1s was examined. A significant amount of the MTA1s was colocalized with the endogenous ER in the cytoplasm only in the ZR-75R cells expressing MTA1s. Interestingly, estradiol rapidly redistributed the ER to the nucleus in ZR-75R/vector cells but not in over 90% of estradiol-activated ZR-75R/MTA1s cells. The observed MTA1s interaction with the ER in the cytoplasm impaired the nuclear functions of ER as ZR-75R/MTA1s cells exhibited a significantly reduced expression of the ER-target genes pS2, c-Myc and cathepsin-D (Dubik and Shiu, 1988; Berry, et al., 1989) (FIG. 5A), indicating that MTA1s may play a role in the development of a hormone-independent phenotype in breast cancer cells. Accordingly, progesterone failed to stimulate PRE-mediated transcription in MTA1s-overexpressing ZR-75R cells (FIG. 5B, right Panel) but not in control cells (FIG. 5B, left Panel). Also, the growth of ZR-75R cells expressing control vector was stimulated by estradiol in an estrogen antagonist ICI 182780-sensitive manner (FIG. 5C). In contrast, ZR-75R cells expressing MTA1s, did not respond to estradiol, implying that these cells represent a hormone-independent phenotype. The expression of MTA1s, significantly enhanced the ability of ZR-75R or MCF-7 cells to grow in an anchorage-independent manner in soft agar (FIGS. 5D, E). Consistent with more aggressive phenotypes, the MTA1s expressing cells were also more tumorigenic in nude mice (FIG. 5F). Furthermore, there was a dramatic increase in MTA1s expression in the tumors over the adjacent normal tissues from the same breast cancer patients in seven out of eight cases. Since MTA1s physically binds to ER and regulates the nuclear functions of ER, the level of MTA1s in a series of retrospective specimens from the ER-positive (tumors with nuclear ER) and ER-negative (tumors without nuclear ER) breast tumors were examined by western blot analysis.

MTA1s levels were significantly upregulated in the ER-negative tumors compared to the ER-positive ones. The quantitation and normalization of the MTA1s signal to the control vinculin indicated a fourfold increase in MTA1s expression in the ER-negative tumors compared to the MTA1s level in ER-positive tumors. As with several anti-peptide antibodies against ER coactivator, including AIBI (Anzick et al., 1997), anti-MTA1s used here did not work well in assays of paraffin-embedded tumor specimens. Considering that MTA1s sequesters ER in the cytoplasm and that MTA1s expression was high in ER-negative tumors, these findings may explain the loss of nuclear ER in breast tumors with elevated MTA1s. These results indicate that MTA1s may promote tumorigenicity of breast tumors without nuclear ER.

A novel pathway that may redirect ER signaling by sequestering it in the cytoplasm and blocking the activation of ER-responsive pathways in the nuclear compartment was identified. The regulation of the cellular localization of ER by MTA1s represents a new mechanism for controlling the nuclear functions of ER, and the deregulation of this pathway may contribute to the progression of breast cancer to more malignant phenotypes.

### Example 3

### MTA1s-Interaction with Bifunctional ATP Sulfurylase/Adenosine 5′-Phosphosulfate Kinase (APS)

The initiation and promotion of breast cancers is regulated by estrogen stimulation of mammary epithelial cell growth. The estrogen receptor, a principal target of estrogen, is found in about 40% of breast tumors at presentation, together with a profile of ER-regulated genes. These tumors are generally responsive to anti-hormonal therapy but eventually develop resistance. Under physiological conditions, estrogen is inactivated by sulfation by estrogen sulfotransferase (EST), and hence, reducing the responsiveness of the target tissue to estrogenic stimulation (Falany 1997). The process of sulfation is entirely dependent on enzymatic synthesis of an activated sulfate donor (3′-phosphoadenosine 5′-phosphosulfate), generated by a bifunctional ATP sulfurylase/adenosine 5′-phosphosulfate (APS) kinase enzyme (Falany 1997). ER negative breast tumors show high level of EST while ER positive tumors have extremely low EST. Accordingly, EST and its inhibitors have also emerged as new targets against hormone dependent breast tumors (Ahmed et al., 2002). However, the molecular pathway that functionally links both ER and estrogen sulfation is yet to be elucidated. This pathway presents a dual molecular target to effectively fight against hormone dependent breast cancer.

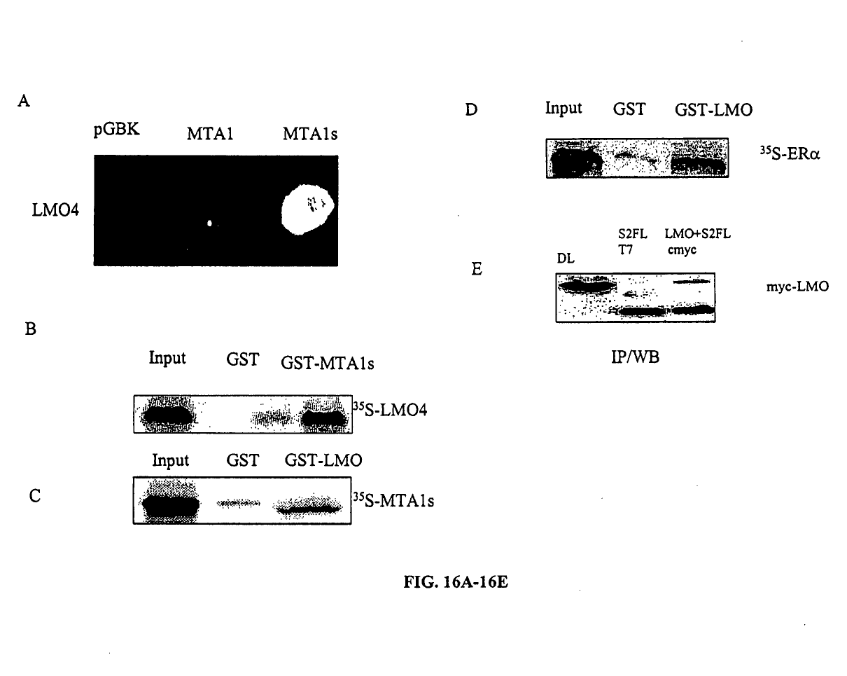

To study the mechanism underlying unique cytoplasmic functions of MTA1s in mammalian cells, a yeast two-hybrid screen was performed to identify MTA1s-interacting proteins, as described above. One of several isolates identified was the bifunctional ATP sulfurylase/adenosine 5′-phosphosulfate (APS) kinase, an enzyme essential for estrogen sulfation (Genebank # NM_{—}005443) (FIGS. 16A-16E). This is an important observation as it supports the idea that the action of MTA1s involves both cytoplasmic ER retention as well as potential modification of estrogen sulfation. Since both EST and MTA1s are known to be upregulated in ER nuclear negative breast tumors, it is likely that MTA1s-interaction with the APS kinase may result in an enhanced estrogen sulfation and estrogen inactivation. Alternatively, MTA1s-APS interaction might deplete APS available for ER sulfation, and thus, prevent estrogen inactivation leading to enhance cellular effects of estrogen in cancer cells. Therefore, certain embodiments of the invention may include targeting the minimum interacting regions of MTA1s and/or APS by a small peptide to modulate the cellular sensitivity to estrogen and selective estrogen-receptor modulators (SERMs).

### Example 4

### MTA1s A Regulation of LMO4 Functions

LIM domain-containing transcriptional regulators are important in the fundamental life processes such as pattern formation and organogenesis in a variety of species, including humans. LMO family members such as LMO1 and LMO2 are oncogene in lymphocytes. While LMO4 controls the developmental functions of several nuclear factors by virtue of LMO4 interactions with these factors while LMO4 might translocate in-and-out of the nucleus (Kenny et al., 1998.). Furthermore, LMO4 has been also shown to upregulated in human breast tumors (Visvader et al., 2001.). In certain embodiments of the invention, methods for identifying LMO4-binding proteins that might affect its functions and activity by influencing the subcellular localization of LMO4 and its ability to interact with other binding partners is contemplated.

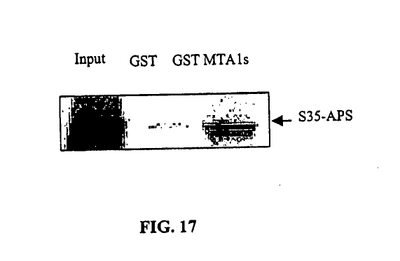

To unravel the mechanism underlying unique cytoplasmic functions of MTA1s in mammalian cells, a yeast two-hybrid screen was performed to identify MTA1s-interacting proteins. One of several isolates was identified as LMO4 (Genebank # NM_{—}006769 (FIG. 17). Thus, MTA1s-interaction with LMO4 may modulate the functions of LMO4 in human cells, and may be involved in tumorigenesis.

### Example 5

### MTA1s, A Modulator of Tamoxifen Resistance

The steroid hormone 17β-estradiol (E2) plays an important role in controlling the expression of genes involved in a wide variety of biological processes, including breast and endometrial cancer progression. The biological effects of estrogen are mediated by its binding to the structurally and functionally distinct estrogen receptors (ERs) ERα and ERβ which mediate E₂ targeted gene transcription, thus promoting cell proliferation. In addition to the genomic actions of ER, nongenomic functions are implicated via activation of intracellular signal transduction pathways in the regulation of cell proliferation by E2. Antiestrogens and selective estrogen receptor modulators have been shown to be effective in controlling the progression of hormone dependent breast tumors because of their antiestrogenic qualities. Many patients with metastatic breast cancer eventually develop resistance to hormonal treatment.

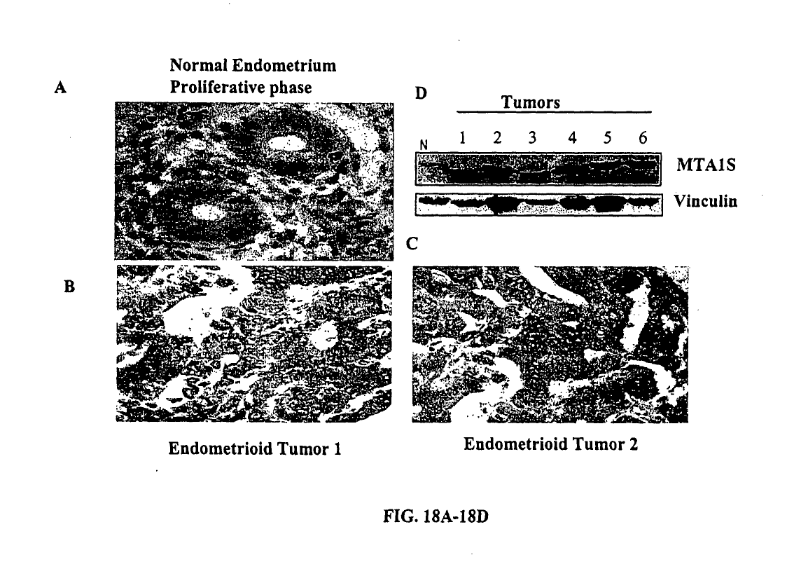

MTA1s is expressed in stromal tissues in normal proliferative endometrium. Our preliminary studies suggest that MTA1s expression is deregulated in endometrial tumors (FIGS. 18A-18D) and immunohistological examination of MTA1s in endometrial tumors suggested that it is predominantly localized in the cytoplasm of epithelial cells as opposed to predominant localization in the stromal of normal proliferative endometrium. Since deregulation of MTA1s hypersensitized breast cancer cells to E₂, by persistent activation of MAPK and promoted tumorigenic phenotype. Earlier studies implicated persistent MAPK signaling as one of the mechanism by which Tamoxifen behaves as a partial agonist in endometrial tissues. The altered expression of MTA1s (stroma vs epithelium) and by its ability to promote nongenomic signaling (i.e., activation of MAPK in an E2 dependent manner) may lead to Tamoxifen resistance. Peptides blocking MTA1s interaction with ER may have therapeutic value in combating Tamoxifen resistance, thus MTA1s may constitute a new target for therapeutic intervention in Tamoxifen resistance.

### Example 6

### MTA1s as a Marker of Tumor Progression

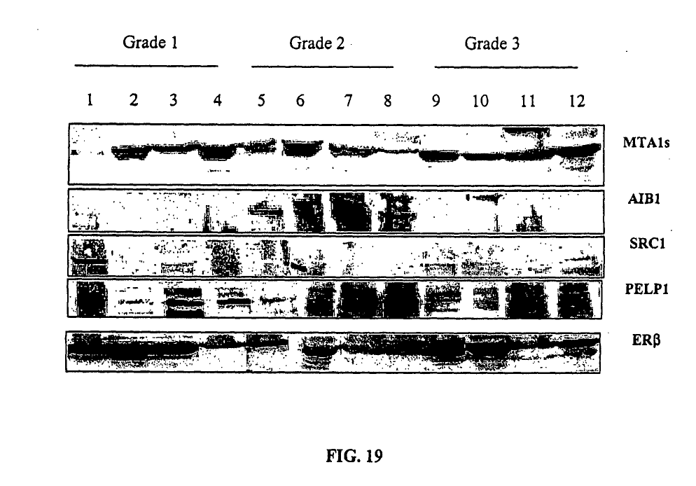

Results suggest that MTA1s expression is highly upregulated in Grade III endometrial tumors compared to grade I and Grade II (FIG. 19). Since grade III tumors exhibit hormonal independent phenotype, expression of MTA1s can potentially be used as a marker of endometrial tumor progression and will have diagnostic value.

### Example 7

### The Significance of Localization of Coactivators in Tamoxifen Resistance

First, a MCF-7 model cell lines will be generated. The stable cell lines will express PELP1 construct which lacks nuclear localization signal and pcDNA vector as a control. The clones will be characterized by western, northern and RT PCR analysis. The localization of PELP1 will be chracterized by confocal microscopic analysis. These cell lines will then be used in testing and characterizing their hormonal responsiveness. ERE reporter gene assays will be used in the presence of various agonist and antagonists. Analysis of signaling pathways may be studied using western blotting in the presence agonist and antagonists. Other methods of analysis may include CHIP analysis using various ERE target sequences, effect of tamoxifen on cell growth and anchorage independence, comparison of E2 and tamoxifen responses between MCF7 and model cell lines, and other methods known to those skilled in the art.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,554,101
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,354,855
U.S. Pat. No. 5,445,941
U.S. Pat. No. 5,582,981
U.S. Pat. No. 5,756,291
U.S. Pat. No. 5,780,610
U.S. Pat. No. 5,792,453
U.S. Pat. No. 5,792,613
U.S. Pat. No. 5,840,867
U.S. Pat. No. 6,004,940
U.S. Pat. No. 6,100,242
U.S. Pat. No. 6,365,361
U.S. Pat. No. 6,506,559
U.S. Pat. No. 6,506,559
U.S. Patent Pub. 20030068821
U.S. Patent Pub. 20030059944
U.S. Patent Pub. 20030056235
U.S. Patent Pub. 20030055020
U.S. Patent Pub. 20030051263
U.S. Patent Pub. 20020173478
U.S. Patent Pub. 20020132788
Ahmed et al., Curr. Med. Chem. 9, 263-273, 2002.
Al. Saati et al., Int J Cancer, 55:651-654, 1993.
Angel et al., Mol. Cell. Biol., 7:2256, 1987.
Angel et al., Cell, 49:729, 1987b.
Angel et al., Mol. Cell. Biol., 7:2256, 1987a.
Anzick et al., Science, 277:965-968, 1997.
Atchison and Perry, Cell, 46:253, 1986.
Atchison and Perry, Cell, 48:121, 1987.
Baichwal and Sugden, In: Gene Transfer, Kucherlapati (ed.), New York, Plenum Press, 117-148, 1986.
Banerji et al., Cell, 27(2 Pt 1):299-308, 1981.
Banerji et al., Cell, 33(3):729-740, 1983.
Barany and Merrifield, Gross and Meienhofer (eds.), Academic Press, New York, 1-284, 1979.
Barnes et al., J. Biol. Chem., 272(17):11510-11517, 1997.
Bellus, J. Macromol. Sci. Pure Appl. Chem., A31(1): 1355-1376, 1994.
Benvenisty and Neshif, Proc. Natl. Acad. Sci. USA, 83(24):9551-9555, 1986.
Berkhout et al., Cell, 59:273-282, 1989.
Berry et al., Proc. Natl. Acad. Sci, 86:1218-1222, 1989.
Bhavsar et al., Genomics, 35(1):11-23, 1996.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Bowman and Bertozzi, Chem. Biol., 6(1):R9-R22., 1999.
Braddock et al., Cell, 58:269, 1989.
Bulla and Siddiqui, J. Virol., 62:1437, 1986.
Campbell and Villarreal, Mol. Cell. Biol., 8:1993, 1988.
Campbell et al., J. Mol. Biol., 180: 1-19, 1984.
Campere and Tilghman, Genes and Dev., 3:537, 1989.
Campo et al., Nature, 303:77, 1983.
Capaldi et al., Biochem. Biophys. Res. Comm., 76:425, 1977.
Celander and Haseltine, J. Virology, 61:269, 1987.
Celander et al., J. Virology, 62:1314, 1988.
Chandler et al., Cell, 33:489, 1983.
Chang et al., Hepatology, 14:134A, 1991.
Chang et al., Mol. Cell. Biol., 9:2153, 1989.
Chatterjee et al., Proc. Natl. Acad Sci. USA, 86:9114, 1989.
Chen and Okayama, Mol. Cell Biol., 7(8):2745-2752, 1987.
Choi et al., Cell, 53:519, 1988.
Coffin, In: Virology, Fields et al., eds., Raven Press, New York, pp. 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Crawford et al., Genomics, 56, 90-97, 1999.
Cripe et al., EMBO J, 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Nail. Acad. Sci. USA, 81:7529-7533, 1984.
Dubik and Shiu, J. Biol. Chem,. 263:12705-12708, 1988.
Edbrooke et al., Mol. Cell. Biol., 9:1908, 1989.
Edlund et al., Science, 230:912-916, 1985.
Elbashir et al., Genes Dev., 5(2):188-200, 2001.
EPO Application No. 320 308
EPO Patent No. 0273085
Ernst et al., Mol. Endocrinol., 5(11):1597-1606, 1991.
Falany, FASEB J., 11:1-2; 206-216, 1997.
Fechheimer et al., Proc. Nail. Acad. Sci. USA, 84:8463-8467, 1987.
Feng and Holland, Nature, 334:6178, 1988.
Ferkol, et al., FASEB J., 7:1081-1091, 1993.
Firak and Subramanian, Mol. Cell. Biol., 6:3667, 1986.
Flototto et al., Horm. Metab. Res., 33(8):451-457, 2001.
Foecking and Hofstetter, Gene, 45(1):101-105, 1986.
Forster and Symons, Cell, 49(2):211-220, 1987.
Fraley et al., Proc. Natl. Acad. Sci. USA, 76:3348-3352, 1979.
Franz et al., Cardioscience, 5(4):235-43, 1994.
Freifelder, Physical Biochem. Apps. Biochem. Molec. Biol., 2Ed. Wm. Freeman and Co., NY, 1982.
Friedmann, Science, 244:1275-1281, 1989.
Fujita et al., Cell, 49:357, 1987.
Gefter et al., Somatic Cell Genet., 3(2):231-6, 1977.
Gerlach et al., Nature (London), 328:802-805, 1987.
Ghosh and Bachhawat, In: Liver Diseases, Targeted Diagnosis and Therapy Using Specific Receptors and Ligands. Wu et al., eds., Marcel Dekker, NY, 87-104, 1991.
Ghosh-Choudhury et al., EMBO J, 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Goding, In: Monoclonal Antibodies: Principles and Practice, 2Ed., Academic Press, FL., 60-66; 71-74, 1986.
Gomez-Foix et al., J. Biol. Chem., 267:25129-25134, 1992.
Goodbourn and Maniatis, Proc. Natl Acad. Sci. USA, 85:1447, 1988.
Goodbourn et al., Cell, 45:601, 1986.
Gopal, Mol. Cell Biol., 5:1188-1190,1985.
Gopal-Srivastava et al., J. Mol. Cell. Biol. 15(12):7081-90, 1995.
Graham and Prevec, Biotechnology, 20:363-390, 1992.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. General Virology, 36:59-74, 1977.
Greene et al., Immunology Today, 10:272, 1989
Grosschedl and Baltimore, Cell, 41:885, 1985.
Grunhaus et al., Seminar in Virology, 200(2):535-546, 1992.
Hammond et al., Nature, 110-119, 2001.
Hammond, et al., Clin. Res. 42:123A, 1994.
Harland and Weintraub, J. Cell Biol., 101: 1094-1099, 1985.
Harlow and Lane, Antibodies: A Laboratory manual, Cold Spring Harbor Laboratory, 1988.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl Acad. Sci. USA, 90:2812-2816, 1993.
Hirochika et al, J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Horwich et al. J. Virol, 64:642-650, 1990.
Huang et al., Cell, 27:245, 1981.
Hug et al., Mol. Cell. Biol., 8:3065, 1988.
Huse, et al., Science, 246:1276 (1989)
Hwang et al., Mol. Cell. Biol., 10:585, 1990.
Imagawa et al., Cell, 51:251, 1987.
Imbra and Karin, Nature, 323:555, 1986.
Imler et al., Mol. Cell Biol., 7:2558, 1987.
Imperiale and Nevins, Mol. Cell. Biol., 4:875, 1984.
Innis et al., PCR Protocols, Academic Press, Inc., San Diego Calif., 1990.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., In: Biotechnology And Pharmacy, Pezzuto et al., eds., Chapman and Hall, New York, 1993.
Johnson et al., Mol. Cell. Biol., 9:3393, 1989.
Jones and Shenk, Cell, 13:181-188, 1978.
Joyce, Nature, 338:217-244, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J. Biol. Chem., 266:3361-3364, 1991.
Kawamoto et al., Mol. Cell. Biol., 8:267, 1988.
Kelly et al., J. Cell. Biol, 129(2):383-96, 1995.
Kenny et al., Proc. Natl. Acad. Sci. USA, 95(19):11257-62, 1998.
Kiledjian et al., Mol. Cell. Biol., 8:145, 1988.
Kim and Cook, Proc. Natl. Acad. Sci. USA, 84(24):8788-8792, 1987.
Kimura et al., Dev. Growth Differ., 39(3):257-65, 1997.
Klaassen and Boles, FASEB J., 11(6):404-418, 1997.
Klamut et al., Mol. Cell. Biol., 10:193, 1990.
Klein et al., Nature, 327:70-73, 1987.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Kriegler and Botchan, In: Eukaryotic Viral Vectors, Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kumar et al., Cell, 51:941-951, 1987.
Kunz et al., Nucl. Acids Res., 17:1121, 1989.
Kyte and Doolittle, J. Mol Biol., 157(1):105-132,1982.
LaPointe et al., Hypertension, 27(3 Pt 2):715-22, 1996.
Larsen et al., Proc Natl. Acad. Sci. USA, 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Lee et al., Nucleic Acids Res., 12:4191 -206, 1984.
Levinson et al., Nature, 295:79, 1982.
Levrero et al., Gene, 101: 195-202, 1991.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Magee et al., J. Cell Biol., 63(2):492-504, 1974.
Majors and Varmus, Proc. Natl. Acad. Sci. USA, 80:5866, 1983.
Mandal et al., J. Biol. Chem., 2001.
Mann et al., Cell, 33:153-159,1983.
Markowitz et al., J. Virol., 62:1120-1124, 1988.
Mazumdar et al., Nature Cell Biol., 3, 30-37, 2001.
McNeall et al., Gene, 76:81, 1989.
Merrifield, Science, 232: 341-347, 1986.
Michel and Westhof, J. Mol. Biol., 216:585-610, 1990.
Miksicek et al., Cell, 46:203, 1986.
Mordacq and Linzer, Genes and Dev., 3:760, 1989.
Moreau et al., Nucl. Acids Res., 9:6047, 1981.
Moss et al., Biol. Chem., 271(49):31688-94, 1996.
Muesing et al., Cell, 48:691, 1987.
Ng et al., Nuc. Acids Res., 17:601, 1989.
Nicolas and Rubinstein, In: Vectors: A survey of molecular cloning vectors and their uses, Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Palmiter et al., Nature, 300:611, 1982.
Paskind et al., Virology, 67:242-248, 1975.
PCT Appl. WO 84/03564
PCT Application No. WO 90/07641
Pech et al., Mol. Cell. Biol., 9:396, 1989.
Pelletier and Sonenberg, Nature, 334:320-325, 1988.
Perales et al., Proc. Natl. Acad. Sci. USA, 91:4086-4090, 1994.
Perez-Stable and Constantini, Mol. Cell. Biol., 10:1116, 1990.
Picard and Schaffner, Nature, 307:83, 1984.
Pietras et al., Oncogene, 10(12):2435-2446, 1995.
Pietras, Cancer Res., 38(4):1019-1030, 1978.
Pignon et al., Hum. Mutat., 3: 126-132, 1994.
Pinkert et al., Genes and Dev., 1:268, 1987.
Ponta et al., Proc. Natl. Acad. Sci. USA, 82:1020, 1985.
Porton et al., Mol. Cell. Biol., 10: 1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Remington's Pharmaceutical Sciences, 15th Edition, Chapter 61, pages 1035-1038 and 1570-1580.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, pp. 467-492, 1988.
Ripe et al., Mol. Cell. Biol., 9:2224, 1989.
Rippe et al., Mol. Cell Biol., 10:689-695, 1990.
Ritchie, J. Biol. Chem., 271(41):25485-25491, 1996.
Rittling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sakai et al., Genes and Dev., 2:1144, 1988.
Sambrook et al., In: Molecular cloning, Cold Spring Harbor Laboratory Press, Cold Spring Harbor, N.Y., 1989.
Sarver et al., Science, 247:1222-1225, 1990.
Satake et al., J. Virology, 62:970, 1988.
Scanlon et al., Proc. Natl. Acad. Sci. USA, 88:10591-10595, 1991.
Schaffner et al., J. Mol. Biol., 201:81, 1988.
Searle et al., Mol. Cell. Biol., 5:1480, 1985.
Shapiro and Senapathy, Nucleic Acid Res., 15:7155-7174, 1987.
Sharp and Marciniak, Cell, 59:229, 1989.
Sharp, Genes Dev., 15:485490, 2001.
Shaul and Ben-Levy, EMBO J., 6:1913, 1987.
Sherman et al., Mol. Cell. Biol., 9:50, 1989.
Simoncini et al., Nature, 407(6803):538-541, 2000.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stewart and Young, In: Solid Phase Peptide Synthesis, 2d. ed., Pierce Chemical Co., 1984.
Stratford-Perricaudet and Perricaudet, In: Human Gene Transfer, Eds, Cohen-Haguenauer and Boiron, Editions John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol, 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (ed.), NY, Plenum Press, 149-188, 1986.
Thiesen et al., J. Virology, 62:614, 1988.
Toh et al., J. Biol. Chem., 269, 229-263, 1994.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. USA, 77:1068, 1980.
Visvader et al., Proc. Natl. Acad. Sci. USA, 98(25):14452-7, 2001.
Wagner et al., Proc. Natl Acad. Sci. USA, 87(9):3410-3414, 1990.
Wang and Calame, Cell, 47:241, 1986.
Weber et al., Cell, 36:983, 1984.
Weinberger et al. Mol. Cell. Biol., 8:988, 1984.
Winoto and Baltimore, Cell, 59:649, 1989.
Wong et al., Gene, 10:87-94, 1980.
Wu and Wu, J. Biol. Chem., 262:4429-4432, 1987.
Wu and Wu, Adv. Drug Delivery Rev., 12:159-167, 1993.
Wu and Wu, Biochemistry, 27:887-892, 1988.
Wu et al., Genomics, 4:560-569, 1989.
Yamauchi-Takihara, et. al., Proc. Natl. Acad. Sci. USA, 86(10):3504-3508, 1989.
Yang et al., Proc. Natl. Acad. Sci. USA, 87:9568-9572, 1990.
Yutzey et al. Mol. Cell. Biol., 9:1397, 1989.
Zelenin et al., FEBS Lett., 280:94-96, 1991.
Ziober and Kramer, J. Biol. Chem., 271(37):22915-22, 1996.

1. An isolated polypeptide comprising an amino acid sequence of at least 5 consecutive amino acids of amino acid sequence 398 to 430 of SEQ ID NO:2.

2. The isolated polypeptide of claim 1, wherein the polypeptide comprises an amino acid sequence of at least 10 consecutive amino acids of the amino acids 398 to 430 of SEQ ID NO:2.

3. The isolated polypeptide of claim 2, wherein the polypeptide comprises an amino acid sequence of at least 15 consecutive amino acids of the amino acids 398 to 430 of SEQ ID NO:2.

4. The isolated polypeptide of claim 3, wherein the polypeptide comprises an amino acid sequence of at least 20 consecutive amino acids of the amino acids 398 to 430 of SEQ ID NO:2.

5. The isolated polypeptide of claim 4, wherein the polypeptide comprises an amino acid sequence of at least 25 consecutive amino acids of the amino acids 398 to 430 of SEQ ID NO:2.

6. The isolated polypeptide of claim 5, wherein the polypeptide comprises an amino acid sequence of at least 30 consecutive amino acids of the amino acids 398 to 430 of SEQ ED NO:2.

7. The isolated polypeptide of claim 6, wherein the polypeptide comprises amino acids 398 to 430 of SEQ ID NO:2.

8. The isolated polypeptide of claim 7, wherein the polypeptide comprises the amino acid sequence of SEQ ID NO:2.

9. An antibody selectively immunoreactive to a MTA1s polypeptide.

10. The antibody of claim 9, wherein the antibody is immunoreactive to the amino acid sequence 398 to 430 of SEQ ID NO:2.

11. The antibody of claim 9, wherein the antibody is a monoclonal antibody.

12. The antibody of claim 9, wherein the antibody is a polyclonal antibody.

13. The antibody of claim 9, wherein the antibody further comprises a detectable label.

14. The antibody of claim 13, wherein the detectable label is selected from the group consisting of a fluorescent label, a chemiluminescent label, a radiolabel and an enzymatic label.

15. A hybridoma cell that produces a monoclonal antibody that is selectively immunoreactive to MTA1s.

16. An isolated nucleic acid comprising a nucleic acid sequence, or complement thereof, encoding an MTA1s polypeptide.

17. The isolated nucleic acid of claim 16, wherein the nucleic acid sequence comprises SEQ ID NO:1.

18. The nucleic acid of claim 16, wherein the nucleic acid is a complementary DNA or RNA.

19. The nucleic acid of claim 18, wherein the nucleic acid further comprises a promoter operably linked to the region, or the complement thereof, encoding the MTA1s polypeptide.

20. The nucleic acid of claim 19, further comprising a polyadenylation signal operably linked to the region encoding the MTA1s polypeptide.

21. The nucleic acid of claim 16, wherein the nucleic acid is a viral vector selected from the group consisting of retrovirus, adenovirus, herpesvirus, vaccinia virus and adeno-associated virus.

22. The nucleic acid of claim 16, wherein the nucleic acid is packaged in a virus particle.

23. The nucleic acid of claim 16, wherein the nucleic acid is packaged in a liposome.

24. An isolated oligonucleotide comprising a sequence of nucleotides that hybridize to a nucleic acid sequence comprising nucleotides 1181 or 1201 of SEQ ID NO:1.

25. The isolated oligonucleotide of claim 24 comprising between about 15 and about 50 consecutive bases.

26. The oligonucleotide of claim 24, further comprising a detectable label.

27. The oligonucleotide of claim 26, wherein the detectable label is selected from the group consisting of a fluorescent label, a chemiluminescent label, a radiolabel and an enzymatic label.

28. An oligonucleotide pair comprising a nucleotide sequence that is within 1000 nucleotides of a splice junction represented by nucleotides 1192 and 1193 of SEQ ID NO:1, wherein the splice junction is flanked by the oligonucleotide pair.

29. The oligonucleotide pair of claim 28, wherein the oligonucleotides comprise about 15 to about 50 consecutive bases of a nucleic acid sequence of SEQ ID NO:1.

30. The oligonucleotide pair of claim 28, wherein the oligonucleotide pair, when used in a DNA amplification reaction, amplifies a DNA segment comprising the splice junction.

31. An expression vector comprising a nucleic acid encoding a negative modulator of an MTA1s polypeptide.

32. The vector of claim 31, wherein the negative modulator of MTA1s is an anti-sense nucleic acid, a ribozyme, an intrabody, or an aptamer.

33. The vector of claim 31, further comprising a promoter operably positioned with respect to a nucleic acid encoding a negative modulator of MTA1s.

34. The vector of claim 33, wherein the promoter is selected from the group consisting of CMV IE, SV40 IE, RSV, β-actin, tetracycline regulatable promoter and ecdysone regulatable promoter.

35. The vector of claim 33, further comprising a polyadenylation signal.

36. The vector of claim 35, wherein the polyadenylation signal is from BGH, thymidine kinase or SV40.

37. A host cell expressing an MTA1s polypeptide as set forth in SEQ ID NO:2.

38. A host cell expressing an polypeptide comprising all or part of amino acids 398 to 430 of SEQ ID NO:2.

39. The host cell of claim 38, wherein the host cell is a stably transfected cell.

40. The host cell of claim 38, wherein the host cell is a transiently transfected cell.

41. The host cell of claim 38, wherein the polypeptide expression is inducible.

42. The host cell of claim 38, wherein polypeptide expression is constitutive.

43. A method of detecting an MTA1s polypeptide in a cell comprising:
i) contacting a cell with an antibody composition that is immunoreactive with a peptide comprising amino acids 398 to 430 of SEQ ID NO:2; and
ii) detecting the antibody composition that is immunoreactive with the cell.

44. The method of claim 43, wherein the antibody composition comprises a monoclonal antibody.

45. The method of claim 43, wherein the antibody composition comprises a polyclonal antibody.

46. A method of restoring hormone sensitivity to a cell comprising contacting a cell with a negative modulator of an MTA1s polypeptide.

47. The method of claim 46, wherein the negative modulator is an antisense molecule, an intrabody, an aptamer, or a small molecule.

48. A method for inhibiting the growth of a cancer cell comprising contacting a cancer cell with a negative modulator of an MTA1s polypeptide.

49. The method of claim 48, wherein the negative modulator of an MTA1s polypeptide is an antibody, an intrabody, an aptamer, an anti-sense molecule, or small molecule.

50. A method of identifying a modulator of an MTA1s polypeptide activity comprising:
(i) providing a cell expressing an MTA1s polypeptide and a steroid hormone receptor responsive reporter construct;
(ii) contacting the cell with a candidate substance; and
(iii) comparing expression from the steroid hormone receptor responsive reporter construct with the expression from a steroid hormone receptor responsive reporter construct observed when the candidate substance is not added, wherein an alteration in expression from a steroid hormone receptor responsive reporter construct indicates that the candidate substance is a modulator of an MTA1s polypeptide.

51. The method of claim 50, wherein the candidate substance is a peptide, peptide mimetic or small molecule.

52. The method of claim 50, wherein the candidate substance is selected from a small molecule library.

53. The method of claim 50, wherein the candidate substance is a protein.

54. The method of claim 50, wherein the candidate substance is a MTA1s analogue.

55. A method of diagnosing hormone insensitive cancer in a subject diagnosed with cancer comprising:
i) obtaining a tissue sample from the subject;
ii) determining the level of MTA1s expression in the tissue sample;
iii) comparing the level of MTA1s expression in the tissue sample to a normal standard control, wherein an increased level of MTA1s expression is diagnostic of hormone insensitivity.

56. The method of claim 55, wherein the level of MTA1s expression is determined by RNA analysis.

57. The method of claim 56, wherein RNA analysis is by a RNAse protection assay.

58. The method of claim 55, wherein the level of MTA1s expression is determined by protein analysis.

59. The method of claim 58, wherein protein analysis is western blot analysis.

60. A method of determining disease prognosis in a subject with cancer comprising:
i) providing a sample of tissue isolated from a cancerous tissue from a subject;
ii) quantifying expression of MTA1s in the sample; and
iii) correlating the quantity of expression of the MTA1s in the sample with a prognosis of the cancer in the subject, wherein higher expression of the MTA1s in the sample correlates with increased likelihood of a poor prognosis.

61. The method of claim 60, wherein quantifying expression of MTA1s in the sample is by RNA analysis.

62. The method of claim 60, wherein quantifying expression of MTA1s in the sample is by protein analysis.

63. The method of claim 60, wherein detection is by RNase protection assay.

64. The method of claim 60, wherein the mRNA is isolated from a cancer cell.
